(19) United States
(12) Patent Application Publication
(10) Pub. No.: US 2026/0108513 A1
(43) Pub. Date: Apr. 23, 2026

(54) Combination of Talazoparib and Enzalutamide in the Treatment of Metastatic Castration-Resistant Prostate Cancer

(71) Applicants: Pfizer Inc., New York, NY (US); Astellas Pharma Inc., Tokyo (JP)

(72) Inventors: Akos Gabor Czibere, Cambridge, MA (US); Dana Ann Kennedy, San Diego, CA (US); Maria Teresa Koehler, Narberth, PA (US); Sandra Jean Meech, Groton, CT (US); Fong Wang, Piedmont, CA (US)

(21) Appl. No.: 19/117,616

(22) PCT Filed: Sep. 29, 2023
(86) PCT No.: PCT/IB2023/059790

§ 371 Date: Apr. 1, 2025

Related U.S. Application Data
(60) Provisional application No. 63/412,520, filed on Oct. 2, 2022.
(60) Provisional application No. 63/434,465, filed on Dec. 21, 2022.
(60) Provisional application No. 63/445,886, filed on Feb. 15, 2023.
(60) Provisional application No. 63/470,761, filed on Jun. 2, 2023.

(51) Int. Cl. A61K 31/5025; A61K 31/4166; A61K 45/06; A61P 35/04

(52) U.S. Cl. CPC A61K 31/5025; A61K 31/4166; A61K 45/06; A61P 35/04

(57) ABSTRACT

This invention relates to talazoparib, or a pharmaceutically acceptable salt thereof, in combination with enzalutamide, or a pharmaceutically acceptable salt thereof, for the treatment of metastatic castration-resistant prostate cancer and methods of increasing survival thereof.

### BACKGROUND

Prostate cancer is the second leading cause of cancer death in men. The androgen receptor (AR) signaling axis, the principal driver of prostate cancer growth, has been targeted by castration and other systemic therapies. Initial treatment for advanced prostate cancer may involve reducing the amount of androgens produced by the body, primarily in the testes. This can be achieved surgically by removal of both testicles (bilateral orchiectomy) or through use of androgen deprivation therapies such as luteinizing hormone-releasing hormone (LHRH) agonist or antagonist drugs, which lower the native production of testosterone (sometimes called “chemical castration”). However, a proportion of tumors progress despite castrate levels of testosterone, at which point the disease is considered castration-resistant. Castration-resistant prostate cancer represents a lethal transition in the progression of prostate cancer, with most patients ultimately succumbing to the disease.

Anti-androgens are thought to suppress androgen activity by a number of different mechanisms. One example of an anti-androgen approved for the treatment of castration-resistant prostate cancer is abiraterone acetate (marketed as Zytiga™), a steroidal CY17A1 inhibitor. One specific class of anti-androgens are androgen receptor inhibitors, also known as androgen receptor signaling inhibitors or androgen receptor antagonists, which are thought to compete with endogenous ligands, androgens, for the androgen receptor. When an antagonist binds to an androgen receptor it is thought to induce a conformational change in the receptor itself that impedes transcription of key androgen regulated genes and therefore inhibits the biological effects of the androgens themselves, such as testosterone and dihydrotestosterone.

The compound, enzalutamide, which is 4-[3-[4-cyano-3-(trifluoromethyl)phenyl]-5,5-dimethyl-4-oxo-2-thioxo-1-imidazolidinyl]-2-fluoro-N-methyl-benzamide (also known as 4-{3-[4-cyano-3-(trifluoromethyl)phenyl]-5,5-dimethyl-4-oxo-2-sulfanylideneimidazolidin-1-yl}-2-fluoro-N-methylbenzamide or also referred to as “RD162” and “MDV3100”) is a non-steroidal androgen receptor inhibitor, having the structure:

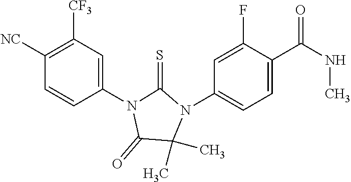 [SMILES: CNC(=O)c1ccc(N2C(=S)N(c3ccc(C#N)c(C(F)(F)F)c3)C(=O)C2(C)C)cc1F]

Enzalutamide, or a pharmaceutically acceptable salt thereof, is disclosed in PCT/US2006/011417, which published on 23 Nov. 2006 as WO 2006/124118, the contents of which are included herein by reference.

Enzalutamide (marketed as Xtandi®) is approved for the treatment of metastatic castration-resistant prostate cancer (“mCRPC”). However, for some subjects, their cancer will relapse or the subjects may develop therapeutic resistance. The mechanisms that underlie such resistance are, to date, not yet fully understood.

Poly (ADP-ribose) polymerase (PARP) engages in the naturally occurring process of deoxyribonucleic acid (DNA) repair in a cell. PARP inhibition has been shown to be an effective therapeutic strategy against tumors associated with mutations in double-strand DNA repair genes by inducing synthetic lethality (Sonnenblick, A., et al., Nat. Rev. Clin. Oncol, 2015, 12(1), 27-4). PARP inhibition is synthetically lethal in cells with homozygous deletions or deleterious alterations, or both, in DNA damage response (DDR) genes involved either directly or indirectly in homologous recombination repair (HRR) (Lord, C J, et al., Science, 2017; 355: 1152-1158).

Talazoparib is a potent, orally available PARP inhibitor, which is cytotoxic to human cancer cell lines harboring gene mutations that compromise deoxyribonucleic acid (DNA) repair, an effect referred to as synthetic lethality, and by trapping PARP protein on DNA thereby preventing DNA repair, replication, and transcription.

The compound, talazoparib, which is (8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one and (8S,9R)-5-fluoro-8-(4-fluorophenyl)-9-(1-methyl-1H-1,2,4-triazol-5-yl)-2,7,8,9-tetrahydro-3H-pyrido[4,3,2-de]phthalazin-3-one (also referred to as “PF-06944076”, “MDV3800”, and “BMN673”) is a PARP inhibitor, having the structure:

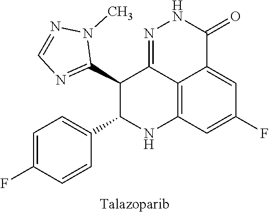 [SMILES: Cn1ncnc1[C@H]1c2n[nH]c(=O)c3cc(F)cc(c23)N[C@@H]1c1ccc(F)cc1]

Talazoparib, and pharmaceutically acceptable salts thereof, including the tosylate salt, are disclosed in International Publication Nos. WO 2010/017055 and WO 2012/054698. Additional methods of preparing talazoparib, and pharmaceutically acceptable salts thereof, including the tosylate salt, are described in International Publication Nos. WO 2011/097602, WO 2015/069851, and WO 2016/019125. Additional methods of treating cancer using talazoparib, and pharmaceutically acceptable salts thereof, including the tosylate salt, are disclosed in International Publication Nos. WO 2011/097334 and WO 2017/075091. Combination treatments using talazoparib, and pharmaceutically acceptable salts thereof, including the tosylate salt, are disclosed in International Publication Nos. WO2019/075032 and WO 2022/200982.

TALZENNA® (talazoparib) (0.25 mg and 1 mg capsules) has been approved in several countries, including the United States, and in the European Union, and is approved or under review with anticipated approvals in other countries for the treatment of adult patients with deleterious or suspected deleterious gBRCAm HER2-negative locally advanced or metastatic breast cancer. Additional capsule strengths, 0.5 mg and 0.75 mg, have been approved in the United States. Talazoparib has shown activity in metastatic castration-resistant prostate cancers with DDR alterations either directly or indirectly associated with HRR (de Bono et al., Lancet Oncol. 2021 September; 22(9):1250-1264). Talazoparib is under development for a variety of human cancers both as a single agent and in combination with other agents.

There remains a need for improved therapies for the treatment of cancers, particularly for the treatment of metastatic castration-resistant prostate cancer. The combinations of the present invention are believed to have one or more advantages, such as an increase in survival, including radiographic progression free survival and overall survival, as compared to treatment of either therapeutic agent alone; increase in survival, including radiographic progression free survival and overall survival, as compared to a patient who has received enzalutamide, or a pharmaceutically acceptable salt thereof, and placebo; greater efficacy as compared to treatment with either therapeutic agent alone; potential to enable an improved dosing schedule; potential to overcome resistance mechanisms, and the like.

### SUMMARY

The present invention provides, in part, methods for administering talazoparib, or a pharmaceutically acceptable salt thereof, and enzalutamide, or a pharmaceutically acceptable salt thereof, in combination therapies, for increasing survival in a subject having metastatic castration-resistant prostate cancer. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to Embodiment 1 of the invention, there is provided a method of increasing survival in a subject having metastatic castration-resistant prostate cancer, comprising: 1) orally administering to the subject talazoparib, or a pharmaceutically acceptable salt thereof, once daily, and 2) orally administering to the subject enzalutamide, or a pharmaceutically acceptable salt thereof, once daily.

According to Embodiment 2 of the invention, there is provided a method of treating metastatic castration-resistant prostate cancer in a subject in need thereof, comprising: 1) orally administering to the subject talazoparib, or a pharmaceutically acceptable salt thereof, once daily; and 2) orally administering to the subject enzalutamide, or a pharmaceutically acceptable salt thereof, once daily, wherein the administration of the talazoparib, or a pharmaceutically acceptable salt thereof, and the administration of the enzalutamide, or a pharmaceutically acceptable salt thereof, increases survival in the subject.

According to Embodiment 3 of the invention, there is provided a combination of talazoparib, or a pharmaceutically acceptable salt thereof, and enzalutamide, or a pharmaceutically acceptable salt or solvate thereof, for use in increasing survival in a subject having metastatic castration-resistant prostate cancer, wherein the talazoparib, or a pharmaceutically acceptable salt thereof, is orally administered to the subject once daily, and the enzalutamide, or a pharmaceutically acceptable salt thereof, is orally administered to the subject once daily

According to Embodiment 4 of the invention, there is provided combination of talazoparib, or a pharmaceutically acceptable salt thereof, and enzalutamide, or a pharmaceutically acceptable salt or solvate thereof, for use in treating a subject having metastatic castration-resistant prostate cancer, wherein the talazoparib, or a pharmaceutically acceptable salt thereof, is orally administered to the subject once daily, and the enzalutamide, or a pharmaceutically acceptable salt thereof, is orally administered to the subject once daily, and further wherein the administration of the talazoparib, or a pharmaceutically acceptable salt thereof, and the administration of the enzalutamide, or a pharmaceutically acceptable salt thereof, increases survival in the subject.

Described below are embodiments of the invention, where for convenience Embodiments 1, 2, 3, and 4 (E1, E2, E3, and E4) are identical to the embodiments provided above.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of the embodiments of the invention and the Examples included herein. It is to be also understood that the terminology used herein is for the purpose of describing specific embodiments only and is not intended to be limiting.

E1 A method of increasing survival in a subject having metastatic castration-resistant prostate cancer, as defined above.

E2 A method of treating metastatic castration-resistant prostate cancer in a subject in need thereof, wherein the treatment increases survival in the subject, as defined above.

E3 A combination for use in increasing survival in a subject having metastatic castration-resistant prostate cancer, as defined above.

E4 A combination for use in treating a subject having metastatic castration-resistant prostate cancer in a subject, wherein administration of the combination increases survival in the subject, as defined above.

E5 A method or combination for use of any one of embodiments 1 to 4, wherein the metastatic castration-resistant prostate cancer is metastatic castration-resistant prostate cancer with or without homologous recombination repair (HRR) gene mutations.

E6 A method or combination for use of any one of embodiments 1 to 5, wherein the talazoparib, or pharmaceutically acceptable salt thereof, and the enzalutamide, or a pharmaceutically acceptable salt thereof, are administered concurrently.

E7 A method or combination for use of any one of embodiments 1 to 6, wherein the subject has not received: 1) systemic cancer treatment for non-metastatic castration-resistant prostate cancer or metastatic castration-resistant prostate cancer; 2) treatment with an androgen receptor signaling inhibitor, a PARP inhibitor, cyclophosphamide, or mitoxantrone for prostate cancer; or 3) treatment with platinum-based chemotherapy within 6 months or any history of disease progression on platinum-based therapy within 6 months.

E8 A method or combination for use of embodiment 7, wherein the subject has not received systemic cancer treatment for non-metastatic castration-resistant prostate cancer or metastatic castration-resistant prostate cancer.

E9 A method or combination for use of embodiment 7, wherein the subject has not been treated with an androgen receptor signalling inhibitor, a PARP inhibitor, cyclophosphamide, or mitoxantrone for prostate cancer.

E10 A method or combination for use of embodiment 7, wherein the subject has not been treated with an androgen receptor signaling inhibitor.

E11 A method or combination for use of any one of embodiments 7, 9 or 10, wherein the androgen receptor signaling inhibitor is a second-generation androgen receptor inhibitor.

E12 A method or combination for use of embodiment 11, wherein the second-generation androgen receptor inhibitor is enzalutamide, apalutamide, darolutamide, or aberatirone acetate.

E13 A method or combination for use of embodiment 12, wherein the second-generation androgen receptor inhibitor is enzalutamide, apalutamide, or darolutamide.

E14 A method or combination for use of embodiment 7, wherein the subject has not received treatment with platinum-based chemotherapy within 6 months or any history of disease progression on platinum-based therapy within 6 months.

E15 A method or combination for use of any one of embodiments 1 to 14, wherein the subject is receiving a gonadotropin releasing hormone analog concurrently or has had a bilateral orchiectomy.

E16 A method or combination for use of embodiment 15, wherein the subject is additionally receiving a gonadotropin releasing hormone analog.

E17 A method or combination for use of embodiment 15 or 16, wherein the gonadotropin releasing hormone analog is a gonadotropin releasing hormone agonist.

E18 A method or combination for use of embodiment 15 or 16, wherein the gonadotropin releasing hormone analog is a gonadotropin releasing hormone antagonist.

E19 A method or combination for use embodiment 15, wherein the subject has had a bilateral orchiectomy.

E20 A method or combination for use of any one of embodiments 1 to 19, wherein the subject has progressive disease as defined by 1 or more of the following: 1) prostate specific antigen progression defined by a minimum of 2 rising prostate specific antigen values from 3 consecutive assessments with an interval of at least 7 days between assessments; 2) soft tissue disease progression as defined by RECIST 1.1.; and 3) bone disease progression defined by Prostate Cancer Clinical Trials Working Group 3 with 2 or more new metastatic bone lesions on a whole body radionuclide bone scan.

E21 A method or combination for use of any one of embodiments 1 to 20, wherein the subject has an Eastern Cooperative Oncology Group (ECOG) performance status s 1.

E22 A method or combination for use of any one of embodiments 1 to 21, wherein the subject is unselected for DNA damage response (DDR) mutation status.

E23 A method or combination for use of any one of embodiments 1 to 22, wherein survival is radiographic progression free survival.

E24 A method or combination for use of embodiment 23, wherein radiographic progression free survival is increased as compared to a subject who has received enzalutamide, or a pharmaceutically acceptable salt thereof, and placebo.

E25 A method or combination for use of embodiment 23, wherein radiographic progression free survival is increased as compared to a subject who has received enzalutamide, or a pharmaceutically acceptable salt thereof.

E26 A method or combination for use of any one of embodiments 1 to 22, wherein survival is overall survival.

E27 A method or combination for use of embodiment 26, wherein overall survival is increased as compared to a subject who has received enzalutamide, or a pharmaceutically acceptable salt thereof, and placebo.

E28 A method or combination for use of embodiment 26, wherein overall survival is increased as compared to a subject who has received enzalutamide, or a pharmaceutically acceptable salt thereof.

E29 A method or combination for use of any one of embodiments 1 to 22, wherein survival is a significantly reduced risk of disease progression or death, as compared to a subject who has received enzalutamide, or a pharmaceutically acceptable salt thereof, and placebo.

E30 A method or combination for use of any one of embodiments 1 to 22, wherein survival is a significantly reduced risk of disease progression or death, as compared to a subject who has received enzalutamide, or a pharmaceutically acceptable salt thereof.

E31 A method or combination for use of embodiment 29, wherein the significantly reduced risk of disease progression or death is a 37% reduction, as compared to a subject who has received enzalutamide, or a pharmaceutically acceptable salt thereof, and placebo.

E32 A method or combination for use of embodiment 29, wherein the significantly reduced risk of disease progression or death is at least a 37% reduction, as compared to a subject who has received enzalutamide, or a pharmaceutically acceptable salt thereof, and placebo.

E33 A method or combination for use of embodiment 30, wherein the significantly reduced risk of disease progression or death is a 37% reduction, as compared to a subject who has received enzalutamide, or a pharmaceutically acceptable salt thereof.

E34 A method or combination for use of embodiment 30, wherein the significantly reduced risk of disease progression or death is at least a 37% reduction, as compared to a subject who has received enzalutamide, or a pharmaceutically acceptable salt thereof.

E35 A method or combination for use of any one of embodiments 1 to 34, wherein the talazoparib, or pharmaceutically acceptable salt thereof, is administered at a dosage equivalent to about 0.1 mg, about 0.25 mg, about 0.35 mg or about 0.5 mg once daily of talazoparib free base.

E36 A method or combination for use of embodiment 35, wherein the talazoparib, or pharmaceutically acceptable salt thereof, is administered at a dosage equivalent to about 0.1 mg once daily of talazoparib free base.

E37 A method or combination for use of embodiment 35, wherein the talazoparib, or pharmaceutically acceptable salt thereof, is administered at a dosage equivalent to about 0.25 mg once daily of talazoparib free base.

E38 A method or combination for use of embodiment 35, wherein the talazoparib, or pharmaceutically acceptable salt thereof, is administered at a dosage equivalent to about 0.35 mg once daily of talazoparib free base.

E39 A method or combination for use of embodiment 38, wherein the subject has moderate renal impairment.

E40 A method or combination for use of embodiment 35, wherein the talazoparib, or pharmaceutically acceptable salt thereof, is administered at a dosage equivalent to about 0.5 mg once daily of talazoparib free base.

E41 A method or combination for use of any one of embodiments 1 to 40, wherein the talazoparib, or pharmaceutically acceptable salt thereof, is talazoparib tosylate.

E42 A method or combination for use of any one of embodiments 1 to 41, wherein the enzalutamide, or pharmaceutically acceptable salt thereof, is administered at a dosage equivalent to about 160 mg once daily of enzalutamide free base.

E43 A method or combination for use of embodiment 42, wherein the dosage of enzalutamide, or a pharmaceutically acceptable salt thereof, is reduced, if enzalutamide, or a pharmaceutically acceptable salt thereof, is dosed concomitantly with a strong CYP2C8 inhibitor.

E44 A method or combination for use of embodiment 42, wherein the dose of enzalutamide, or a pharmaceutically acceptable salt thereof, is reduced to 80 mg once daily.

E45 A method or combination for use of embodiment 42, wherein the dose of enzalutamide, or a pharmaceutically acceptable salt thereof, is increased if the enzalutamide is dosed concomitantly with a CYP3A4 inducer.

E46 A method or combination for use of embodiment 42, wherein the dose of enzalutamide, or a pharmaceutically acceptable salt thereof, is increased to 240 mg daily.

E47 A method or combination for use of any one of embodiments 1 to 46, wherein the enzalutamide, or a pharmaceutically acceptable salt thereof, is a free base.

E48 A method or combination for use of any one of embodiments 1 to 47, wherein the method comprises administering a further anti-cancer agent.

E49 A method or combination for use of embodiment 48, wherein the further anti-cancer agent is selected from the group consisting of an anti-tumor agent, an anti-angiogenesis agent, a signal transduction inhibitor, and an antiproliferative agent.

E50 A method or combination for use of any of the preceeding embodiments, wherein the subject is a human.

E51 A method or combination for use of embodiment 50, wherein the human is an adult.

Each of the embodiments described herein may be combined with any other embodiment(s) described herein not inconsistent with the embodiment(s) with which it is combined.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

As used herein, the singular form “a”, “an”, and “the” include plural references unless indicated otherwise. For example, “a” substituent includes one or more substituents.

As used herein, the term “about” when used to modify a numerically defined parameter (e.g., the dose of a talazoparib, or a pharmaceutically acceptable salt thereof) means that the parameter may vary by as much as 10% below or above the stated numerical value for that parameter. For example, a dose of about 5 mg means 5 mg±10%, i.e., it may vary between 4.5 mg and 5.5 mg.

As used herein, terms, including, but not limited to, “agent”, “composition, “compound”, “drug”, “medicine” and “therapeutic agent” may be used interchangeably to refer to compounds included in the methods and uses of the present invention, such as an anti-androgen, androgen receptor signaling inhibitors, androgen deprivation therapy, talazoparib, and enzalutamide.

For purposes of the present invention, “DDR mutation(s)”, “DDR alteration(s)”, “HRR mutation(s)”, “HRR alteration(s)”, and “HRR gene alteration(s)” refer to alterations/mutations in genes involved directly or indirectly in homologous recombination repair (HRR). Though not as scientifically robust as the phrase “DNA damage response”, is commonly understood that “DDR” may also be referred to as “DNA damage repair” or “DNA repair”. “DDR-deficient” refers to gene mutations associated with deficiencies in deoxyriboneclueic acid (DNA) damage repair. A “DDR-deficient patient population” or an “HRR-deficient patient population” is a patient population with gene mutations associated with deficiencies in deoxyriboneclueic acid (DNA) damage repair. DDR is a network of pathways which have evolved to repair damaged DNA. These include mismatch repair, base excision repair, and homologous recombination repair (HRR) among others. HRR is particularly important in maintaining genomic integrity given its high fidelity in repairing double-strand DNA breaks. Inhibition of PARP results in accumulation of single-strand DNA breaks and in DNA stress due to PARP trapping, which ultimately culminates in double-strand DNA breaks. Hence, PARP inhibitors are selectively lethal to cancer cells deficient in HRR− this is an example of synthetic lethality, a mechanism whereby deficiency in function of one gene or gene product has little effect alone but is toxic in combination with deficiency in function of a second gene or gene product. DDR-HRR genes include, but are not 20 limited to, ATM, ATR, BRCA1, BRCA2, CHEK2, FANCA, MLH1, MRE11A, NBN, PALB2, and RAD51C. A deficiency in homologous recombination repair may be determined using next generation sequencing (NGS).

For purposes of the present invention, “radiographic” and “imaging-based” may be used interchangeably. For example, “radiographic” progression is the same as “imaging-based” progression; radiographic PFS is the same as imaging-based PFS (ibPFS); and rPFS is the same as ibPFS.

As used herein, “systemic therapy” for mCRPC are drugs or therapeutic agents used to manage mCRPC. The medicines or drugs are referred to as systemic therapies because they circulate throughout the body to attack cancer cells wherever they may be located in the body.

### Anti-Androgens

As used herein, the terms “anti-androgen” and “anti-androgens” refer to compounds which prevent androgens, for example testosterone and dihydrotestosterone (DHT) and the like, from mediating their biological effects in the body. Anti-androgens may act by one or more of the following hormonal mechanisms of action such as blocking and/or inhibiting and/or modulating the androgen receptor (AR); inhibiting androgen production; suppressing androgen production; degrading the AR, inhibiting nuclear translocation, inhibiting binding of the AR to nuclear DNA, and the like. Anti-androgens include, but are not limited to, steroidal androgen receptor inhibitors (for example, cyproterone acetate, spironolactone, megestrol acetate, chlormadinone acetate, oxendolone, and osaterone acetate), non-steroidal androgen receptor inhibitors (for example, enzalutamide, bicalutamide, nilutamide, flutamide, topilutamide, apalutamide and darolutamide), androgen synthesis inhibitors, androgen receptor degraders and the like. Anti-androgens include androgen receptor inhibitors or androgen receptor signaling inhibitors, terms which are used interchangeably. Androgen receptor inhibitors can be determined by methods known to those of skilled in the art, for example using in vitro assays and/or cellular ligand binding assays and/or gene expression assays such as those disclosed in Tran C., et al., Science, 2009, 324, 787-790.

First generation androgen receptor signaling inhibitors include bicalutamide, nilutamide, or flutamide.

Second-generation androgen receptor signaling inhibitors include enzalutamide, apalutamide and darolutamide.

A further second-generation androgen receptor signaling inhibitor is abiraterone, or a pharmaceutically acceptable salt or solvate thereof, such as abiraterone acetate (marketed as Zytiga™), a steroidal CY17A1 inhibitor which is disclosed in U.S. Pat. No. 5,604,213 which published on 18 Feb. 1997, the contents of which are incorporated herein by reference. This second-generation AR inhibitor prevents androgen biosynthesis.

An example of an androgen receptor inhibitor is N-desmethyl enzalutamide:

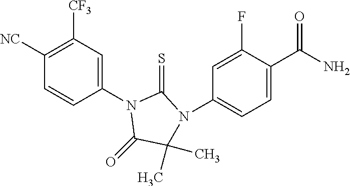 [SMILES: CC1(C)C(=O)N(c2ccc(C#N)c(C(F)(F)F)c2)C(=S)N1c1ccc(C(N)=O)c(F)c1]

or a pharmaceutically acceptable salt or solvate thereof, also known as 4-[3-[4-cyano-3-(trifluoromethyl)phenyl]-5,5-dimethyl-4-oxo-2-thioxoimidazolidin-1-yl]-2-fluorobenzamide; or MII; which is disclosed in PCT/US2010/025283, which published on 2 Sep. 2010 as WO 2010/099238, the contents of which are included herein by reference.

An example of an androgen receptor inhibitor is apalutamide (marketed as ERLEADA®):

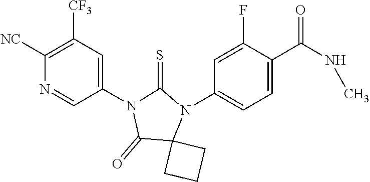 [SMILES: CNC(=O)c1ccc(N2C(=S)N(c3cnc(C#N)c(C(F)(F)F)c3)C(=O)C23CCC3)cc1F]

or a pharmaceutically acceptable salt or solvate thereof, also known as ARN-509; or 4-{7-[6-cyano-5-(trifluoromethyl)pyridine-3-yl]-8-oxo-6-thioxo-5,7-diazaspiro[3,4]octan-5yl}-2-fluoro-N-methylbenzamide; which is disclosed in PCT/US2007/007485, which published on 8 Nov. 2007 as WO 2007/126765, the contents of which are included herein by reference. In one embodiment, the androgen receptor inhibitor useful in the present invention is a pharmacologically active metabolite of apalutamide, or a pharmaceutically acceptable salt or solvate thereof.

An example of an androgen receptor inhibitor is darolutamide (marketed as NUBEQA®):

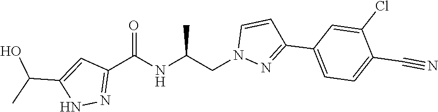 [SMILES: CC(O)c1cc(C(=O)N[C@@H](C)Cn2ccc(-c3ccc(C#N)c(Cl)c3)n2)n[nH]1]

or a pharmaceutically acceptable salt or solvate thereof, also known as N-[(2S)-1-[3-(3-chloro-4-cyanophenyl)-1H-pyrazol-1-yl]propan-2-yl]-5-(1-hydroxyethyl)-1H-pyrazole-3-carboxamide which is disclosed in PCT/F12010/000065, which published on 5 May 2011 as WO 2011/051540, the contents of which are included herein by reference.

An example of an androgen receptor inhibitor is bicalutamide:

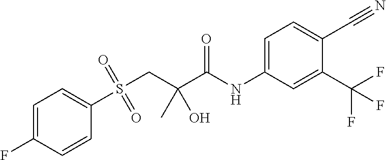 [SMILES: CC(O)(CS(=O)(=O)c1ccc(F)cc1)C(=O)Nc1ccc(C#N)c(C(F)(F)F)c1]

or a pharmaceutically acceptable salt or solvate thereof, marketed as Casodex©, which is disclosed in U.S. Pat. No. 4,636,505, published on 13 Jan. 1987, the contents of which are included herein by reference.

An example of an androgen receptor inhibitor is nilutamide (marketed as Nilandron®), or a pharmaceutically acceptable salt or solvate thereof.

An example of an androgen receptor inhibitor is flutamide (marketed as Eulexin®), or a pharmaceutically acceptable salt or solvate thereof.

Unless indicated otherwise, all references herein to the anti-androgens and androgen receptor inhibitors includes references to salts, solvates, hydrates and complexes thereof, and to solvates, hydrates and complexes of salts thereof, including polymorphs, stereoisomers, and isotopically labeled versions thereof.

### Androgen Deprivation Therapy

Androgen deprivation therapy, also called ADT, uses surgery or medicines to lower the levels of androgens made by the testicles.

An example of surgical ADT is bilateral orchiectomy.

Examples of medicinal ADT include a luteinizing hormone-releasing hormone (LHRH) agonist, a LHRH antagonist, a gonadotropin releasing hormone (GnRH) agonist and a GnRH antagonist.

Other examples of medicinal androgen deprivation therapy include leuprolide (also known as leuprorelin, for example Lupron or Eligardor Viadur and the like); buserelin (for example Suprefact); gonadorelin; goserelin (for example Zoladex); histrelin (for example Vantas); nafarelin; triptorelin (for example Trelstar); deslorelin; fertirelin; abarelix (for example Plenaxis); cetrorelix; degarelix (for example Firmagon); ganirelix; ozarelix; elagolix (for example Orilissa); relugolix; and linzagolix.

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

### Administration and Dosing

As used herein, the terms, “subject” and “patient,” are used interchangeably, to refer to any animal, including mammals. Mammals according to the invention include canine, feline, bovine, caprine, equine, ovine, porcine, rodents, lagomorphs, primates, humans and the like. In an embodiment, humans are suitable subjects. In an embodiment, a “subject” or “patient” is an adult human.

A “subject” or “patient” according to the combination of this invention may have imaging performed while receiving therapy to evaluate their response to treatment. Response criteria, specifically Response Evaluation Criteria in Solid Tumors version 1.1 (RECIST 1.1), are standardized and may be used at different time points to classify response into the categories, such as complete response (CR), partial response (PR), stable disease (SD), or disease progression. At the trial level, categorical responses for all patients are summated into image-based trial endpoints.

A “subject” or “patient” according to the combination of this invention may have: 1) histologically or cytologically confirmed adenocarcinoma of the prostate without small cell or signet cell features; 2) asymptomatic or mildly symptomatic metastatic castration-resistant prostate cancer; 3) DNA damage repair (DDR) deficiency as assessed centrally by a next-generation sequencing (NGS) biomarker mutation panel that contains DDR genes likely to sensitize to PARP inhibition; 4) surgically or medically castrated, with serum testosterone s 50 ng/dL (s 1.73 nmol/L) at screening; 5) ongoing androgen deprivation therapy with a gonadotropin releasing hormone (GnRH) agonist or antagonist for patients who have not undergone bilateral orchiectomy; 6) metastatic disease in bone documented on bone scan or in soft tissue documented on CT/MRI scan; 7) progressive disease at study entry in the setting of medical or surgical castration as defined by one or more of the following three criteria: i) prostate specific antigen (PSA) progression defined by a minimum of two rising PSA values from 3 assessments with an interval of at least 7 days between assessments; ii) soft tissue disease progression as defined by RECIST 1.1; and iii) bone disease progression defined by Prostate Cancer Clinical Trials Working Group 3 (PCWG3) with 2 or more new metastatic bone lesions on a whole body radionuclide bone scan; 8) ongoing bisphosphonate or denosumab use; 9) Eastern Cooperative Oncology Group (ECOG) performance status s 1; and 10) life expectancy 12 months as assessed by the investigator.

A “subject” or “patient” according to the combination of this invention may be unselected for DNA damage response (DDR) mutation status or, interchangeably, unselected for HRR gene alteration status. “Unselected for DDR mutation status” and “unselected for HRR gene alteration status” mean that the subject is not selected for treatment based on DDR mutation status or HRR gene alteration status. In other words, all subjects having metastatic castration-resistant prostate cancer are eligible for treatment with the combination of the invention regardless of DDR mutation status or HRR gene alteration status.

As used herein, the term “cancer” refers to or describes the physiological condition in a patient of subject that is typically characterized by unregulated cell growth. Cancer refers to any malignant and/or invasive growth or tumor caused by abnormal cell growth. The term “metastatic” as it relates to cancer, includes but is not limited to, a cancer that has spread from the place in which it started to other parts of the body, a recurrence from the original primary cancer after remission, and a second primary cancer that is a new primary cancer in a subject with a history of previous cancer of different type from latter one. Those skilled in the art will be able to recognize and diagnose metastatic cancer in a patient.

“Treat” or “treating” a metastatic cancer, such as mCRPC, as used herein means to administer a combination therapy according to the present invention to a subject or patient having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The term “treatment” or “therapy,” as used herein, unless otherwise indicated, refers to the act of treating as “treating” is defined immediately above. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (2009)).

In one embodiment, the treatment achieved by a combination of the invention is increasing survival in the subject.

In one embodiment, the increase in survival is measured by any of the following: progression free survival (PFS), radiographic PFS (rPFS), and overall survival (OS). PFS, rPFS and OS are clinically meaningful endpoints for measuring an increase in survival for patients treated with the combination of the present invention. PFS is the length of time during and after the treatment of a disease, such as cancer, that a patient lives with the disease but it does not get worse. OS is the length of time from either the date of diagnosis or the start of treatment for a disease, such as cancer, that patients diagnosed with the disease are still alive. In a clinical trial, measuring the PFS and OS are ways to see how well a new treatment works. For purposes of the clinical trial described here, rPFS refers to the time from the date of randomization to first objective evidence of radiographic progression assessed in soft tissue per Response Evaluation Criteria in Solid Tumors version 1.1 (RECIST 1.1) or in bone (upon subsequent confirmation) per Prostate Cancer Clinical Trials Working Group (PCWG3) guidelines by Blinded Independent Central Review (BICR), or death, whichever occurs first. For purposes of the clinical trial described here, OS refers to the time from the date of randomization to death due to any cause.

In one embodiment, the progression free survival is increased as compared to a subject who has received enzalutamide, or a pharmaceutically acceptable salt thereof, and placebo. In one embodiment, the progression free survival is increased as compared to a subject who has received enzalutamide, or a pharmaceutically acceptable salt thereof, and placebo. For purposes of the invention, treatment with placebo means that a patient having metastatic castration-resistant prostate cancer is not being treated with talazoparib.

In one embodiment, the radiographic progression free survival is increased as compared to a subject who has received enzalutamide, or a pharmaceutically acceptable salt thereof, and placebo. In one embodiment, the radiographic progression free survival is increased as compared to a subject who has received enzalutamide, or a pharmaceutically acceptable salt thereof, and placebo. For purposes of the invention, treatment with placebo means that a patient having metastatic castration-resistant prostate cancer is not being treated with talazoparib.

An “amount” for use and for treating a subject refers to an amount that provides, in single or multiple doses, alone, or in combination with one or more other agents, a detectable response of any duration of time (transient, medium or long term), a desired outcome in or an objective or subjective benefit to a subject of any measurable or detectable degree or for any duration of time (e.g., for hours, days, months, years, in remission or cured). Such amounts typically are effective to ameliorate a disease, or one, multiple or all adverse effects/symptoms, consequences or complications of the disease, to a measurable extent, although reducing or inhibiting a progression or worsening of the disease, or providing stability (i.e., not worsening) state of the disease, is considered a satisfactory outcome. The term “therapeutically effective amount” also means an amount of an agent, alone, or in combination with one or more other agents, effective for producing a desired therapeutic effect upon administration to a subject, for example, to stem the growth, or result in the shrinkage, of a cancerous tumor. In reference to the treatment of cancer, a therapeutically effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression.

As used herein, “ameliorate” refers to any reduction in the extent, severity, frequency, and/or likelihood of a symptom or clinical sign characteristic of a particular disease. “Symptom” refers to any subjective evidence of disease or of a subject's condition.

In one embodiment, the amount or daily dosage of talazoparib, or a pharmaceutically acceptable salt thereof, and preferably a tosylate thereof, to be administered to a subject is equivalent to about 0.1 mg to about 1 mg once daily of talazoparib free base. In an embodiment, talazoparib or a pharmaceutically acceptable salt thereof, and preferably a tosylate thereof, is administered at a daily dosage equivalent to about 0.1 mg once daily of talazoparib free base; to about 0.25 mg once daily of talazoparib free base; to about 0.35 mg once daily of talazoparib free base; to about 0.5 mg once daily of talazoparib free base; to about 0.75 mg once daily of talazoparib free base; or to about 1 mg once daily of talazoparib free base. In an embodiment, talazoparib or a pharmaceutically acceptable salt thereof, and preferably a tosylate thereof, is administered at a daily dosage equivalent to about 0.1 mg once daily of talazoparib free base; to about 0.25 mg once daily of talazoparib free base; to about 0.35 mg once daily of talazoparib free base; or to about 0.5 mg once daily of talazoparib free base. In an embodiment, talazoparib or a pharmaceutically acceptable salt thereof and preferably a tosylate thereof, is administered at a daily dosage equivalent to about 0.1 mg once daily of talazoparib free base. In an embodiment, talazoparib or a pharmaceutically acceptable salt thereof and preferably a tosylate thereof, is administered at a daily dosage equivalent to about 0.25 mg once daily of talazoparib free base. In an embodiment, talazoparib or a pharmaceutically acceptable salt thereof and preferably a tosylate thereof, is administered at a daily dosage equivalent to about 0.35 mg once daily of talazoparib free base. In an embodiment, talazoparib or a pharmaceutically acceptable salt thereof and preferably a tosylate thereof, is administered at a daily dosage equivalent to about 0.5 mg once daily of talazoparib free base. In an embodiment, talazoparib or a pharmaceutically acceptable salt thereof and preferably a tosylate thereof, is administered at a daily dosage equivalent to about 0.75 mg once daily of talazoparib free base. In an embodiment, talazoparib or a pharmaceutically acceptable salt thereof and preferably a tosylate thereof, is administered at a daily dosage equivalent to about 1 mg once daily of talazoparib free base.

In one embodiment, the amount or daily dosage of talazoparib, or a pharmaceutically acceptable salt thereof, and preferably a tosylate thereof, to be administered to a subject is about 0.1 mg to about 1 mg once daily of talazoparib free base or equivalent. In an embodiment, talazoparib or a pharmaceutically acceptable salt thereof, and preferably a tosylate thereof, is administered at a daily dosage of about 0.1 mg once daily of talazoparib free base or equivalent; to about 0.25 mg once daily of talazoparib free base or equivalent; to about 0.35 mg once daily of talazoparib free base or equivalent; to about 0.5 mg once daily of talazoparib free base or equivalent; to about 0.75 mg once daily of talazoparib free base or equivalent; or to about 1 mg once daily of talazoparib free base or equivalent. In an embodiment, talazoparib or a pharmaceutically acceptable salt thereof, and preferably a tosylate thereof, is administered at a daily dosage of about 0.1 mg once daily of talazoparib free base or equivalent; to about 0.25 mg once daily of talazoparib free base or equivalent; to about 0.35 mg once daily of talazoparib free base or equivalent; or to about 0.5 mg once daily of talazoparib free base or equivalent. In an embodiment, talazoparib or a pharmaceutically acceptable salt thereof and preferably a tosylate thereof, is administered at a daily dosage of about 0.1 mg once daily of talazoparib free base or equivalent. In an embodiment, talazoparib or a pharmaceutically acceptable salt thereof and preferably a tosylate thereof, is administered at a daily dosage of about 0.25 mg once daily of talazoparib free base or equivalent. In an embodiment, talazoparib or a pharmaceutically acceptable salt thereof and preferably a tosylate thereof, is administered at a daily dosage of about 0.35 mg once daily of talazoparib free base or equivalent. In an embodiment, talazoparib or a pharmaceutically acceptable salt thereof and preferably a tosylate thereof, is administered at a daily dosage of about 0.5 mg once daily of talazoparib free base or equivalent. In an embodiment, talazoparib or a pharmaceutically acceptable salt thereof and preferably a tosylate thereof, is administered at a daily dosage of about 0.75 mg once daily of talazoparib free base or equivalent. In an embodiment, talazoparib or a pharmaceutically acceptable salt thereof and preferably a tosylate thereof, is administered at a daily dosage of about 1 mg once daily of talazoparib free base or equivalent.

Dosage amounts provided herein refer to the dose of the free base form of talazoparib, or are calculated as the free base equivalent of an administered talazoparib salt form. For example, a dosage or amount of talazoparib, such as 0.25, 0.35 mg or 0.5 mg refers to the free base equivalent.

In one embodiment, enzalutamide is dosed in accordance with the XTANDI© US approved label with a daily dose of 160 mg once daily. Dosage adjustments of enzalutamide, in accordance with full XTANDI© prescribing information may be readily determined by one of ordinary skill in the art, such as if the enzalutamide is to be dosed in concomitantly with a strong CYP2C8 inhibitor then the dose of enzalutamide should be reduced in accordance with the full prescribing information, such as to 80 mg once daily; or alternatively if the enzalutamide is to be dosed concomitantly with a CYP3A4 inducer then the dose of enzalutamide should be increased in accordance with the full prescribing information, such as to 240 mg daily.

In a preferred embodiment, enzalutamide, or a pharmaceutically acceptable salt thereof, is administered at a daily dosage of about 160 mg once daily. Dosage amounts provided herein refer to the dose of the free base form of enzalutamide, or are calculated as the free base equivalent of an administered enzalutamide salt form. For example, a dosage or amount of enzalutamide, such as 160 mg, refers to the free base or equivalent.

The recommended dose of talazoparib is 0.5 mg administered orally once daily in combination with enzalutamide 160 mg orally once daily, until disease progression or unacceptable toxicity occurs. This dosage regimen may be adjusted to provide the optimal therapeutic response. For example, the dose may be proportionally reduced or increased as indicated by the exigencies of the therapeutic situation. To manage adverse reactions, consider interruption of treatment with or without dose reduction based on severity and clinical presentation. The 0.35 mg, 0.25 mg and 0.1 mg capsules are available for dose reduction.

For patients with mCRPC and moderate renal impairment, (CLcr 30-59 mL/min) the recommended dose of talazoparib is 0.35 mg once daily in combination with enzalutamide 160 mg orally once daily. For patients with severe renal impairment (CLcr 15-29 mL/min), the recommended dose of talazoparib is 0.25 mg once daily in combination with enzalutamide 160 mg orally once daily.

For patients with mCRPC, reduce the talazoparib dose to 0.35 mg once daily in combination with enzalutamide 160 mg orally once daily when coadministered with certain P-gp inhibitors, such as itraconazole, amiodarone, carvedilol, clarithromycin, itraconazole, and verapamil. When the P-gp inhibitor is discontinued, increase the talazoparib dose (after 3-5 half-lives of the P-gp inhibitor) to the dose used prior to the initiation of the P-gp inhibitor.

The compounds of the invention may be administered orally. Oral administration may involve swallowing, so that the compound enters the gastrointestinal tract, or buccal or sublingual administration may be employed by which the compound enters the bloodstream directly from the mouth.

In a preferred embodiment, the daily dose of talazoparib, or a pharmaceutically acceptable salt thereof, is administered orally.

In a preferred embodiment, the daily dose of enzalutamide, or a pharmaceutically acceptable salt thereof, is administered orally.

Talazoparib, or a pharmaceutically acceptable salt, may be present in a pharmaceutical composition which includes a pharmaceutically acceptable excipient. “Pharmaceutically acceptable excipient” refers to a component that may be included in the compositions described herein, is physiologically suitable for pharmaceutical use, and causes no significant adverse effects nor therapeutic effects to a subject. The term ‘excipient’ is used herein to describe any ingredient other than the compound(s) of the invention. The choice of excipient will to a large extent depend on factors such as the mode of administration, the effect of the excipient on solubility and stability, and the nature of the dosage form.

The amount of talazoparib, or a pharmaceutically acceptable salt, in the pharmaceutical compositions can be any amounts disclosed herein.

The compounds of the method, use or combination of the present invention may be formulated prior to administration. The formulation will preferably be adapted to the particular mode of administration. These compounds may be formulated with pharmaceutically acceptable excipients as known in the art and administered in a wide variety of dosage forms as known in the art. Dosage unit forms or pharmaceutical compositions suitable for oral administration include, but are not limited to tablets, capsules, such as gelatin capsules, pills, powders, granules, aqueous and nonaqueous oral solutions and suspensions, packaged in containers adapted for subdivision into individual doses.

In another embodiment, the dosage of a compound or pharmaceutical composition described herein can vary within the range depending upon the dosage form employed and the route of administration utilized. In another embodiment, an amount of a compound or pharmaceutical composition described herein administered to a subject can be dependent upon factors known to a skilled artisan, including bioactivity and bioavailability of the compound (e.g., half-life and stability of the compound in the body), chemical properties of the compound (e.g., molecular weight, hydrophobility and solubility), route and frequency of administration, and the like. Further, it will be understood that the specific dose of a pharmaceutical composition comprising a compound as disclosed herein can depend on a variety of factors including physical condition of the subject (e.g., age, gender, weight), and medical history of the subject (e.g., medications being taken, health condition other diseases or disorders). The precise dose of a pharmaceutical composition administered to a subject can be determined by methods known to a skilled artisan such as a pharmacologist, or an anesthesiologist.

Repetition of the administration or dosing regimens may be conducted as necessary to achieve the desired reduction or diminution of cancer cells. A “continuous dosing schedule”, as used herein, is an administration or dosing regimen without dose interruptions, e.g., without days off treatment. Repetition of 28-day treatment cycles without dose interruptions between the treatment cycles is an example of a continuous dosing schedule. In an embodiment, the compounds of the combination of the present invention can be administered in a continuous dosing schedule. In an embodiment, the compounds of the combination of the present invention can be administered concurrently in a continuous dosing schedule.

### Therapeutic Methods and Uses

The methods and combination therapies of the present invention are useful for treating mCRPC. The methods and combination therapies of the present invention are useful for treating mCRPC with or without DDR mutations or HRR gene alterations.

In one embodiment, the disclosure provides a method of increasing survival in a subject having metastatic castration-resistant prostate cancer, comprising: 1) orally administering to the subject talazoparib, or a pharmaceutically acceptable salt thereof, once daily, and 2) orally administering to the subject enzalutamide, or a pharmaceutically acceptable salt thereof, once daily.

In one embodiment, the disclosure provides a method of increasing survival in a subject having metastatic castration-resistant prostate cancer, comprising 1) orally administering to the subject talazoparib, or a pharmaceutically acceptable salt thereof, once daily, in combination with 2) orally administering to the subject enzalutamide, or a pharmaceutically acceptable salt thereof, once daily.

In one embodiment, the disclosure provides a method of treating metastatic castration-resistant prostate cancer in a subject in need thereof, comprising: 1) orally administering to the subject talazoparib, or a pharmaceutically acceptable salt thereof, once daily; and 2) orally administering to the subject enzalutamide, or a pharmaceutically acceptable salt thereof, once daily, wherein the administration of the talazoparib, or a pharmaceutically acceptable salt thereof, and the administration of the enzalutamide, or a pharmaceutically acceptable salt thereof, increases survival in the subject.

In one embodiment, the disclosure provides a method of treating metastatic castration-resistant prostate cancer in a subject in need thereof, comprising: 1) orally administering to the subject talazoparib, or a pharmaceutically acceptable salt thereof, once daily, in combination with 2) orally administering to the subject enzalutamide, or a pharmaceutically acceptable salt thereof, once daily, wherein the combination increases survival in the subject.

The term “combination”, as used herein, unless otherwise indicated, means a combination of agents that is administered closely enough in time to affect treatment of the subject. The combination of the invention may be administered concurrently (i.e., simultaneously) or sequentially. Examples of “in combination” include, but are not limited to, “concurrent administration,” “co-administration,” “simultaneous administration,” “sequential administration” and “administered simultaneously”. The combination of the invention may be co-administered in the same formulation. The combination of the invention may be administered concurrently—(i.e., simultaneously) in separate formulations. The combination of the invention may be administered sequentially, i.e., talazoparib is administered first, followed by enzalutamide after a specific duration of time, such as one hour; or enzalutamide is administered first, followed by talazoparib after a specific duration of time, such as one hour. The combination of the invention is preferably administered concurrently.

In one embodiment, the disclosure provides a combination of talazoparib, or a pharmaceutically acceptable salt thereof, and enzalutamide, or a pharmaceutically acceptable salt or solvate thereof, for use in increasing overall survival in the treatment of metastatic castration-resistant prostate cancer in a subject.

In another aspect, this invention relates to the use of talazoparib, or a pharmaceutically acceptable salt thereof, and enzalutamide, or a pharmaceutically acceptable salt thereof, in the manufacture of a medicament for increasing overall survival in the treatment of metastatic castration-resistant prostate cancer in a subject.

In one embodiment, the combination therapy is administered to a subject that has not received: 1) prior systemic cancer treatment for non-metastatic castration-resistant prostate cancer or metastatic castration-resistant prostate cancer; 2) prior treatment with an androgen receptor signaling inhibitor, a PARP inhibitor, cyclophosphamide, or mitoxantrone for prostate cancer; or 3) prior treatment with platinum-based chemotherapy within 6 months from the last dose or any history of disease progression on platinum-based therapy within 6 months from the last dose.

### Further Therapeutic Agents

In one embodiment, the methods and combination therapies of the present invention may additionally comprise administering a further anti-cancer agent, such as an anti-tumor agent, an anti-angiogenesis agent, a signal transduction inhibitor and an antiproliferative agent. In some such embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, androgen deprivation therapy and anti-androgens.

In one embodiment, the methods and combination therapies of the present invention may additionally comprise administering a further active agent, wherein the further active agent is androgen deprivation therapy.

In one embodiment, the androgen deprivation therapy is a luteinizing hormone-releasing hormone (LHRH) agonist, an LHRH antagonist, a gonadotropin-releasing hormone (GnRH) agonist or GnRH antagonist.

In one embodiment, the androgen deprivation therapy is a GnRH agonist or GnRH antagonist.

In one embodiment, the androgen deprivation therapy is a GnRH agonist.

In one embodiment, the androgen deprivation therapy is a GnRH antagonist.

In one embodiment, the androgen deprivation therapy is a LHRH agonist or LHRH antagonist.

In one embodiment, the androgen deprivation therapy is a LHRH agonist.

In one embodiment, the androgen deprivation therapy is a LHRH antagonist.

In one embodiment, the androgen deprivation therapy is selected from the group consisting of leuprolide (also known as leuprorelin, for example Lupron or Eligardor Viadur and the like); buserelin (for example Suprefact); gonadorelin; goserelin (for example Zoladex); histrelin (for example Vantas); nafarelin; triptorelin (for example Trelstar); deslorelin; fertirelin; abarelix (for example Plenaxis); cetrorelix; degarelix (for example Firmagon); ganirelix; ozarelix; elagolix (for example Orilissa); relugolix; and linzagolix.

In one embodiment, the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix.

In one embodiment, the androgen deprivation therapy is selected from the group consisting of leuprolide, goserelin and degaralix.

In one embodiment, the androgen deprivation therapy is leuprolide. In some embodiments the leuprolide is administered intramuscularly at a dose of about 7.5 mg every month, or about 22.5 mg every three months, or about 30 mg every four months.

In one embodiment, the androgen deprivation therapy is leuprolide. In some embodiments the leuprolide is administered subcutaneously at a dose of about 7.5 mg every month, or about 22.5 mg every three months, or about 30 mg every four months, or about 45 mg every six months, or about 65 mg every 12 months.

In one embodiment, the androgen deprivation therapy is goserelin. In some embodiments the goserelin is administered subcutaneously at a dose of about 3.6 mg every month, or about 10.8 mg every three months.

In one embodiment, the androgen deprivation therapy is degarelix. In some embodiments the degarelix is administered intramuscularly at an initial dose of about 240 mg, which initial dose may be optionally divided into several smaller doses, for example 2 doses of about 120 mg, followed by a maintenance dose of about 80 mg every month.

In one embodiment of the methods and combination therapies of the present invention, the regimen includes a further active agent, wherein the further active agent is etoposide. In some embodiments, the etoposide is administered intravenously in accordance with the approved label, for example at a dose of from 50 to 100 mg/m² once a day on days 1 to 5; or from 5 to 100 mg/m² once a day on days 1, 3 and 5. In one example etoposide may be administered at a dose from 80 to 120 mg/m², on days 1, 2 and 3 of each 21-day cycle for 1, 2, 3, 4, 5 or 6 cycles.

### Example

### Study Design

This was an international, Phase 3, randomized, double-blind, two-part study for mCRPC patients, TALAPRO-2 (NCT03395197).

Part 1 was open-label and non-randomized and evaluated the safety, tolerability, and pharmacokinetic (PK) of talazoparib (Tala) in combination with enzalutamide (Enza). Nineteen mCRPC patients were enrolled to establish the appropriate starting dose of talazoparib in combination with enzalutamide for Part 2. The starting dose in Part 2 was talazoparib/placebo 0.5 mg/day (mg QD) in combination with enzalutamide 160 mg/day. Patients with moderate renal impairment at screening had a reduced talazoparib/placebo starting dose of 0.35 mg/day.

Part 2 was randomized, double-blind, and placebo-controlled and evaluated the efficacy and safety of talazoparib in combination with enzalutamide compared with placebo in combination with enzalutamide. Patients were randomized 1:1 to receive either talazoparib or matching placebo in combination with open-label enzalutamide. Randomization was stratified by prior novel hormonal therapy or taxane-based chemotherapy, e.g., abiraterone, orteronel, or docetaxel for castration-sensitive prostate cancer (yes/no) and DDR mutational status, also referred to as, homologous recombination repair gene alteration status (deficient vs. non deficient/unknown). Stratification factors were specified by the investigator, and recorded in the interactive web response system (IWRS) before randomization and used for the stratified analysis of the primary efficacy endpoint. However, as the IWRS grouped non-deficient and unknown status, a secondary stratified analysis based on homologous recombination repair gene alteration status derived from the clinical database was also performed and used to identify the non-deficient subpopulation.

Genomic screening to identify alterations in DDR genes was optional for patients in Part 1, but it was required for randomization in Part 2. Mutational status was determined by testing for the presence of mutations in defined DDR genes likely to sensitize to PARP inhibition using next generation sequencing (NGS) based gene panel test. Prior to randomization, patients consented to provide solid tumor tissue (de novo or archival) and/or blood-based samples, which were prospectively assessed for the alteration status of DNA damage response genes directly or indirectly involved in homologous recombination repair (BRCA1, BRCA2, PALB2, ATM, ATR, CHEK2, FANCA, RAD51C, NBN, MLH1, MRE11A, CDK12) using FoundationOne® and/or FoundationOneLiquid® CDx. Of the 805 enrolled patients, tumor tissue for informing stratification was available from 804 (99.9%) patients, with 114 (14.2%) patients also having blood-based-testing available for circulating tumor DNA (ctDNA). One patient was enrolled based on ctDNA results only. For exploratory analysis, available blood-based samples (not collected in China) were retrospectively tested using FoundationOneLiquid® CDx to determine the status of patients with a prospectively-assessed status of unknown.

Study treatment (including enzalutamide) continued until radiographic/imaging-based progression was determined by (BICR) (Part 2) or local review (Part 1) unless in the opinion of the investigator the patient was still deriving benefit at this time, or an adverse event leading to permanent discontinuation, or patient decision to discontinue treatment, or death.

There were two patient cohorts in Part 2: first the all-corners cohort, then the DDR-deficient cohort. The results from the all-corners cohort unselected for HRR gene alterations, e.g, patients with and without tumor HRR gene alterations, in Part 2 of the study is provided in this Example. DDR-deficient cohort is still ongoing.

### Inclusion Criteria:

1. Histologically or cytologically confirmed adenocarcinoma of the prostate without small cell or signet cell features.
2. Asymptomatic or mildly symptomatic metastatic castration resistant prostate cancer (mCRPC) (score on BPI-SF Question #3 must be <4).
3. For enrollment into Part 2 only (optional in Part 1): assessment of DDR mutation status.
4. Consent to a saliva sample collection for a germline comparator unless prohibited by local regulations or ethics committee decision (optional for patients in Part 1).
5. Surgically or medically castrated, with serum testosterone s 50 ng/dL (s 1.73 nmol/L) at screening.
6. Metastatic disease in bone documented on bone scan or in soft tissue documented on CT/MRI scan.
7. Progressive disease at study entry in the setting of medical or surgical castration as defined by 1 or more of the following 3 criteria (prostate-specific antigen only or imaging-based):
   a. Prostate specific antigen (PSA) progression defined by a minimum of 2 rising PSA values from 3 consecutive assessments with an interval of at least 7 days between assessments.
   b. Soft tissue disease progression as defined by RECIST 1.1.
   c. Bone disease progression defined by Prostate Cancer Clinical Trials Working Group 3 (PCWG3) with 2 or more new metastatic bone lesions on a whole body radionuclide bone scan.
8. Ongoing bisphosphonate or denosumab use prior to Day 1 (Part 1) or randomization (Part 2) is allowed but not mandatory.
9. Eastern Cooperative Oncology Group (ECOG) performance status s 1.
10. Life expectancy 12 months as assessed by the investigator.
11. Able to swallow the study drug and have no known intolerance to study drugs or excipients.
12. Must agree to use a condom when having sex with a partner from the time of the first dose of study drug through 4 months after last dose of study treatment. Must also agree for female partner of childbearing potential to use an additional highly effective form of contraception from the time of the first dose of study treatment through 4 months after last dose of study treatment when having sex with a non pregnant female partner of childbearing potential.
13. Must agree not to donate sperm from the first dose of study drug to 4 months after the last dose of study drug.
14. Evidence of a personally signed and dated informed consent document (and molecular prescreening consent if appropriate) indicating that the patient [or a legally acceptable representative/legal guardian] has been informed of all pertinent aspects of the study.
15. Willing and able to comply with scheduled visits, treatment plan, laboratory tests, and other study procedures.

### Exclusion Criteria:

1. Any prior systemic cancer treatment initiated in in the non metastatic CRPC and mCRPC disease state.
2. Patients whose only evidence of metastasis is adenopathy below the aortic bifurcation.
3. Prior treatment with second-generation androgen receptor inhibitors (enzalutamide, apalutamide, and darolutamide), a PARP inhibitor, cyclophosphamide, or mitoxantrone for prostate cancer.
4. Prior treatment with platinum-based chemotherapy within 6 months (from the last dose) prior to Day 1 (Part 1) or randomization (Part 2), or any history of disease progression on platinum-based therapy within 6 months (from the last dose).
5. Treatment with cytotoxic chemotherapy, biologic therapy including sipuleucel T, or radionuclide therapy received in the castration-sensitive prostate cancer is NOT exclusionary if discontinued in the 28 days prior to Day 1 (Part 1) or randomization (Part 2).
6. Prior docetaxel and abiraterone or orteronel in the castration-sensitive setting was NOT exclusionary.
7. Treatment with any investigational agent within 4 weeks before Day 1 (Part 1) or randomization (Part 2).
8. Prior treatment with opioids for pain related to either primary prostate cancer or metastasis within 28 days prior to Day 1 (Part 1) or randomization (Part 2).
9. Current use of potent P-gp inhibitors within 7 days prior to Day 1 (Part 1) or randomization (Part 2).
10. Major surgery (as defined by the investigator) within 2 weeks before Day 1 (Part 1) or randomization (Part 2), or palliative localized radiation therapy within 3 weeks before randomization (Part 2).
11. Clinically significant cardiovascular disease
12. Significant renal dysfunction as defined by any of the following laboratory abnormalities:
   a. Renal: eGFR <30 mL/min/1.73 m2 by the MDRD equation (available via www.mdrd.com).
13. Patients enrolled in Part 1 only: Moderate renal impairment (eGFR 30-59 mL/min/1.73 m2) at screening.
14. Significant hepatic dysfunction as defined by any of the following laboratory abnormalities on screening labs:
   a. Total serum bilirubin >1.5 times the upper limit of normal (ULN) (≥3×ULN for patients with documented Gilbert syndrome or for whom indirect bilirubin concentrations suggest an extrahepatic source of elevation).
   b. Aspartate aminotransferase (AST) or alanine aminotransferase (ALT) >2.5 times ULN (>5×ULN if liver function abnormalities are due to hepatic metastasis).
   c. Albumin <2.8 g/dL
15. Absolute neutrophil count <1500/μL, platelets <100,000/μL, or hemoglobin <9 g/dL (may not have received growth factors or blood transfusions within 14 days before obtaining the hematology values at screening).
16. Known or suspected brain metastasis or active leptomeningeal disease.
17. Symptomatic or impending spinal cord compression or cauda equina syndrome.
18. Any history of myelodysplastic syndrome, acute myeloid leukemia, or prior malignancy except any of the following:
   a. Carcinoma in situ or non melanoma skin cancer
   b. Any prior malignancies ≥3 years before randomization with no subsequent evidence of recurrence or progression regardless of the stage.
   c. Stage 0 or Stage 1 cancer <3 years before randomization that has a remote probability of recurrence or progression in the opinion of the investigator
19. Gastrointestinal disorder affecting absorption.
20. Fertile male subjects who are unwilling or unable to use highly effective methods of contraception for the duration of the study and for 4 months after the last dose of investigational product.
21. Investigator site staff members directly involved in the conduct of the study and their family members, site staff members otherwise supervised by the investigator, or patients who are Pfizer employees, including their family members, directly involved in the conduct of the study.
22. Other acute or chronic medical (concurrent disease, infection, or comorbidity) or psychiatric condition including recent (within the past year) or active suicidal ideation or behavior or laboratory abnormality that interferes with ability to participate in the study, may increase the risk associated with study participation or investigational product administration, or may interfere with the interpretation of study results and, in the judgment of the investigator, would make the patient inappropriate for entry into this study.
23. History of seizure or any condition that may predispose to seizure (eg, prior cortical stroke, significant brain trauma). Also, history of loss of consciousness or transient ischemic attack within 12 months of randomization (Part 2).

### Statistical Analysis:

Approximately 750 patients were enrolled in the all-corners cohort. For the primary comparison in the all-corners population, 333 ibPFS events (based on BICR) provided 85% power to detect a hazard ratio of 0.696 using a 1-sided stratified log-rank test at a significance level of 0.0125 (to maintain overall type I error at or below 1-sided 0.025, alpha for ibPFS by BICR was split equally between the all-corners [Cohort 1] and forthcoming molecularly selected Cohort 2; 1-sided alpha of 0.0125 for each).

Overall survival was tested only if the ibPFS showed statistically significant improvement, in a hierarchical stepwise procedure to preserve the overall type I error. Other endpoints had no adjustment for multiplicity.

Calculation of sample size and power for ibPFS included the following assumptions: median ibPFS would be 16 months for the placebo+enzalutamide arm and 23 months for the talazoparib+enzalutamide group for the all-corners population; approximately 15% of the all-corners population would harbor homologous recombination repair gene alterations.

Time-to-event endpoints were compared between treatment arms using a stratified log-rank test. Hazard ratios and associated 95% 2-sided confidence intervals (Cis) were estimated by a Cox proportional hazards model. Median time-to-event endpoints were estimated by the Kaplan-Meier method and 95% CIs were based on the Brookmeyer-Crowley method. Missing or partial dates were imputed as specified per protocol. Other missing data were not imputed.

### Patient Population:

The primary population for evaluating efficacy endpoints as well as patient characteristics was the intent-to-treat (ITT) population. This population included all patients who were randomized, with treatment assignment designated according to the randomization, regardless of whether patients received study treatment.

The safety analysis population consisted of all patients who received at least one dose of study treatment (talazoparib/placebo or enzalutamide) and was based on the actual treatment received. This population was the primary population for evaluating safety.

Between 7 Jan. 2019 and 17 Sep. 2020, a total of 805 patients were enrolled and randomized into the all-corners cohort (402 to the talazoparib+enzalutamide group and 403 to the placebo+enzalutamide group, intent-to-treat population). 398 patients in the talazoparib+enzalutamide group and 401 patients in the placebo+enzalutamide group received study treatment (all-corners safety population). Data cut-off was Aug. 16, 2022.

The patient treatment phase disposition is summarized in Table 1 below.

TABLE 1
Patient Disposition by Study Treatment Phase (Safety Population)

| | Talazoparib + | | Placebo + | |
|---|---|---|---|---|
| | Enzalutamide | | Enzalutamide | |
| Treatment Phase | (N = 398) | | (N = 401) | |
| Talazoparib/Placebo*: | 397 | (99.7%) | 400 | (99.8%) |
| Ongoing | 152 | (38.2%) | 120 | (29.9%) |
| Discontinued | 245 | (61.6%) | 280 | (69.8%) |
| Adverse Event | 69 | (17.3%) | 43 | (10.7%) |
| Death | 8 | (2.0%)** | 10 | (2.5%) |
| Lost to follow-up | 0 | | 1 | (0.2%) |
| Progressive disease | 76 | (19.1%) | 123 | (30.7%) |
| Protocol deviation | 0 | | 1 | (0.2%) |
| Withdrawal by patient | 29 | (7.3%) | 30 | (7.5%) |
| No longer meets eligibility criteria | 1 | (0.3%) | 0 | |
| Global deterioration of health | 44 | (11.1%) | 46 | (11.5%) |
| status | | | | |
| No longer clinically benefiting | 14 | (3.5%) | 21 | (5.2%) |
| Other | 4 | (1.0%) | 5 | (1.2%) |
| Enzalutamide: | 397 | (99.7%) | 400 | (99.8%) |
| Ongoing | 170 | (42.7%) | 124 | (30.9%) |
| Discontinued | 227 | (57.0%) | 276 | (68.8%) |
| Adverse Event | 37 | (9.3%) | 36 | (9.0%) |
| Death | 8 | (2.0%) | 11 | (2.7%) |
| Lost to follow-up | 0 | | 1 | (0.2%) |
| Progressive disease | 84 | (21.1%) | 123 | (30.7%) |
| Withdrawal by patient | 30 | (7.5%) | 31 | (7.7%) |
| No longer meets eligibility criteria | 1 | (0.3%) | 0 | |
| Global deterioration of health | 48 | (12.1%) | 47 | (11.7%) |
| status | | | | |
| No longer clinically benefiting | 15 | (3.8%) | 22 | (5.5%) |
| Other | 4 | (1.0%) | 5 | (1.2%) |

*Two patients, one in each treatment group, who received enzalutamide but not talazoparib/placebo were excluded from these data.
**Discontinuation due to death was reported for one patient in error. The patient discontinued both talazoparib and enzalutamide due to the same adverse event on the same date.

Baseline demographic/disease characteristics were well-balanced. The representativeness of the patients is shown in Table 2.

TABLE 2
Representativeness of Study Participants
Category

| Disease under investigation | Metastatic castration-resistant prostate cancer |
|---|---|
| Special considerations related to: | |
| Age | Prevalence increases steeply with age, with |
| | the highest incidence in men >65 years of age |
| Race or ethnicity | African-American men have higher incidence |
| | and mortality rates for prostate cancer |
| | compared with White men |
| Geography | Throughout the world, prostate cancer |
| | incidence and mortality rates vary widely |
| | between countries, with mortality rates in 2020 |
| | between 7.8 and 13.7 per 100,000 in Europe, |
| | 8.3 in North America and some of the lowest |
| | mortality rates in Asia (4.6 per 100,000 in East |
| | Asia) |
| Prior treatment | Many men with metastatic castration-resistant |
| | prostate cancer will have received prior |
| | treatment for castration-sensitive disease with |
| | androgen deprivation therapy plus a hormonal |
| | therapy, such as abiraterone or enzalutamide, |
| | possibly in combination with docetaxel |
| Genetic alterations | More than 20% of men with metastatic |
| | prostate cancer are reported to have |
| | alterations in DNA damage repair genes |
| | involved either directly or indirectly in |
| | homologous recombination repair, the most |
| | common being BRCA2 (approximately 10%) |
| Overall representativeness of | The age distribution of men in the present trial |
| this trial | was consistent with that expected, with the |
| | majority of patients aged >65 years. The |
| | proportion of Black or African American |
| | patients who underwent randomization was |
| | small (2.0%). Men were enrolled from diverse |
| | geographic locations including North America |
| | (15%), Europe/United Kingdom (38%), Asia |
| | (30% in China and Japan), and the rest of the |
| | world (17%). More than half of men received |
| | prior anti-androgen therapy with approximately |
| | 20% receiving prior docetaxel. Alterations in |
| | DNA damage repair genes were reported in |
| | 20.7% of patients, consistent with prior reports |

Table 3 shows the baseline patient demographics and disease characteristics (all-corners intention-to-treat population), which were well-balanced between treatment arms.

TABLE 3
Baseline Patient Demographics and Disease Characteristics
(All-Comers Intention-to-Treat Population)

| | Talazoparib + | | Placebo + | |
|---|---|---|---|---|
| | Enzalutamide | | Enzalutamide | |
| Characteristic | (N = 402) | | (N = 403) | |
| Median age (range) - yr | 71 | (66-76) | 71 | (65-76) |
| Race | | | | |
| White | 243 | (60.4) | 255 | (63.3) |
| Black or African American | 11 | (2.7) | 5 | (1.2) |
| Asian | 127 | (31.6) | 120 | (29.8) |
| Multiracial | 0 | | 1 | (0.2) |
| Other* | 2 | (0.5) | 1 | (0.2) |
| Not reported | 19 | (4.7) | 21 | (5.2) |
| Renal impairment† | | | | |
| Normal or mild | 344 | (85.6) | 347 | (86.1) |
| Moderate | 42 | (10.4) | 41 | (10.2) |
| Median baseline serum PSA | 18.2 | (6.9-59.4) | 16.2 | (6.4-53.4) |
| (range) - μg/L | | | | |
| Median baseline circulating | 1 | (0-7) | 1 | (0-6) |
| tumor cell count (range) - | | | | |
| cells per 7.5 mL of blood | | | | |
| Gleason score** | | | | |
| <8 | 117 | (29.1) | 113 | (28.0) |
| ≥8 | 281 | (69.9) | 283 | (70.2) |
| Initial M stage at | | | | |
| primary diagnosis† | | | | |
| M0 | 172 | (42.8) | 185 | (45.9) |
| M1 | 204 | (50.7) | 193 | (47.9) |
| MX | 22 | (5.5) | 22 | (5.5) |
| Disease site | | | | |
| Bone (including with | 349 | (86.8) | 342 | (84.9) |
| soft tissue component) | | | | |
| Lymph node | 147 | (36.6) | 167 | (41.4) |
| Visceral (lung) | 45 | (11.2) | 61 | (15.1) |
| Visceral (liver) | 12 | (3.0) | 16 | (4.0) |
| Other soft tissue | 37 | (9.2) | 33 | (8.2) |
| ECOG performance status | | | | |
| 0 | 259 | (64.4) | 271 | (67.2) |
| 1 | 143 | (35.6) | 132 | (32.8) |
| Androgen deprivation | | | | |
| therapy at baseline | | | | |
| Bilateral orchiectomy | 24 | (6.0) | 27 | (6.7) |
| Androgen deprivation therapy | 378 | (94.0) | 376 | (93.3) |
| Prior taxane-based | 86 | (21.4) | 93 | (23.1) |
| chemotherapy (docetaxel)*** | | | | |
| Prior treatment with novel | 23 | (5.7) | 27 | (6.7) |
| hormonal agent | | | | |
| Abiraterone§ | 21 | (5.2) | 25 | (6.2) |
| Tissue source for prospective | | | | |
| homologous recombination | | | | |
| repair gene alteration testing | | | | |
| Tumor tissue | 345 | (85.8) | 345 | (85.6) |
| Tumor tissue and blood | 57 | (14.2) | 57 | (14.1) |
| (ctDNA) | | | | |
| Blood (ctDNA) only | 0 | | 1 | (0.2) |
| Homologous recombination | | | | |
| repair gene alteration status | | | | |
| at baseline by IWRS (for | | | | |
| prospective stratification) | | | | |
| Deficient | 85 | (21.1) | 84 | (20.8) |
| Non-deficient or Unknown¶ | 317 | (78.9) | 319 | (79.2) |
| Homologous recombination | | | | |
| repair gene alteration | | | | |
| status derived from the | | | | |
| clinical database | | | | |
| Deficient | 85 | (21.1) | 82 | (20.3) |
| Non-deficient | 207 | (51.5) | 219 | (54.3) |
| Unknown¶ | 110 | (27.4) | 102 | (25.3) |
| BRCA1/2 alteration | 27 | (6.7) | 32 | (7.9) |

Data are no. (%) unless otherwise indicated.
*American Indian, Alaska Native, Native Hawaiian, or Other Pacific Islander.
**Not reported for the remaining patients.
***All received docetaxel.
§The remaining two patients in each treatment arm received orteronel.
¶Prospective “unknown” status for homologous recombination repair gene alteration status primarily reflects technical failure of FoundationOne ® testing due to limitations in sample quality or purity.
ctDNA denotes circulating tumor DNA, ECOG Eastern Cooperative Oncology Group, IWRS, Interactive Web Response System, and PSA, prostrate-specific antigen.

Biomarker status was prospectively informed by tissue for 99.9% of patients. Of 805 patients, 804 had tissue available for prospective testing of homologous recombination repair gene status (1 patient had ctDNA results only). Table 4 shows the source of tumor DNA for assessment and baseline HRR gene status.

TABLE 4
Source of Tumor DNA for Assessment and Baseline
Homologous Recombination Repair Gene Status

| Tissue source for | Talazoparib plus | Placebo plus | |
|---|---|---|---|
| prospective HRR gene | enzalutamide | enzalutamide | |
| alteration testing, n (%) | (N = 402) | (N = 403) | |
| Tumor tissue | 347 (86.3) | 347 | (86.1) |
| Tumor tissue and blood | 57 (14.2) | 57 | (14.1) |
| (circulating tumor DNA) | | | |
| Blood (circulating tumor | 0 | 1 | (0.2) |
| DNA) only | | | |

Data are no. (%) unless otherwise indicated

By prospective testing, 426 (52.9%) had no homologous recombination repair gene alterations detected (non-deficient), 167 (20.7%) had alterations detected (deficient), and 212 (26.3%) were of unknown alteration status (Table 3). BRCA alterations were detected in 6.7% (n=27) in the talazoparib+enzalutamide group and 7.9% (n=32) in the placebo+enzalutamide arm. Table 5 includes a summary of homologous recombination repair gene alterations and shows that HRR gene alterations were well-balanced between treatment arms. Exploratory analysis, including retrospective ctDNA results to resolve the status of prospectively unknown patients, found that 547 (68.0%) were of non-deficient alteration status, 214 (26.6%) were deficient, and 44 (5.5%) were unknown. All results were based on the 4RS unless otherwise stated.

TABLE 5
Summary of homologous recombination repair gene alterations
(all-comers intention-to-treat population)

| | Talazoparib plus | | Placebo plus | |
|---|---|---|---|---|
| | enzalutamide | | enzalutamide | |
| Gene alterations | (N = 402) | | (N = 403) | |
| Number of participants with | 85 | (21.1) | 82 | (20.3) |
| one or more alterations in | | | | |
| the corresponding gene, no. | | | | |
| (%) | | | | |
| CDK12 | 23 | (5.7) | 29 | (7.2) |
| BRCA2 | 23 | (5.7) | 28 | (6.9) |
| ATM | 23 | (5.7) | 14 | (3.5) |
| CHEK2 | 6 | (1.5) | 5 | (1.2) |
| BRCA1 | 5 | (1.2) | 4 | (1.0) |
| MLH1 | 4 | (1.0) | 1 | (0.2) |
| NBN | 4 | (1.0) | 1 | (0.2) |
| PALB2 | 3 | (0.7) | 2 | (0.5) |

| | | | |
|---|---|---|---|
| ATR | 0 | 3 | (0.7) |

| | | | | |
|---|---|---|---|---|
| FANCA | 1 | (0.2) | 4 | (1.0) |
| MRE11A | 1 | (0.2) | 1 | (0.2) |
| RAD51C | 1 | (0.2) | 1 | (0.2) |
| Number of participants with | 7 | (1.7) | 10 | (2.5) |
| one or more alterations in | | | | |
| two or more genes, no. (%) | | | | |
| ATM/BRCA2 | 1 | (0.2) | 2 | (0.5) |

| | | | |
|---|---|---|---|
| ATM/CDK12 | 0 | 2 | (0.5) |

| | | | |
|---|---|---|---|
| ATM/MLH1 | 1 | (0.2) | 0 |

| | | | |
|---|---|---|---|
| ATR/BRCA2 | 0 | 1 | (0.2) |
| ATR/BRCA2/CDK12 | 0 | 1 | (0.2) |

| BRCA1/BRCA2/PALB2 | 1 | (0.2) | 0 | |
|---|---|---|---|---|
| BRCA1/MRE11A/PALB2 | 1 | (0.2) | 0 | |
| BRCA2/CDK12 | 1 | (0.2) | 1 | (0.2) |

| | | | |
|---|---|---|---|
| BRCA2/CHEK2 | 0 | 1 | (0.2) |
| BRCA2/MLH1 | 0 | 1 | (0.2) |
| BRCA2/NBN | 0 | 1 | (0.2) |

| | | | |
|---|---|---|---|
| CDK12/CHEK2 | 1 | (0.2) | 0 |
| CDK12/MLH1 | 1 | (0.2) | 0 |

### Key Efficacy Results & Supportive Findings:

The primary endpoint was BICR-assessed rPFS, also referred to as ibPFS by BICR, per RECIST 1.1 and PCWG3. Based on the data cutoff on 16 Aug. 2022, the number of events in the talazoparib+enzalutamide group was 151/402 and the number of events in the placebo+enzalutamide group was 191/403. The median follow-up for rPFS was 24.9 months and 24.6 months for the talazoparib+enzalutamide and placebo+enzalutamide groups, respectively. The observed stratified hazard ratio (talazoparib+enzalutamide vs. placebo+enzalutamide) for the primary endpoint was 0.627 (95% CI: [0.506, 0.777]; one-sided P-value <0.0001; two-sided P-value <0.0001) in favor of talazoparib+enzalutamide. Median rPFS was not estimable (NE)/not reached (NR) (95% CI: [27.5, NE/not reached]) months for the talazoparib+enzalutamide group vs. 21.9 months (95% CI: [16.6, 25.1]) months for the placebo+enzalutamide group. Treatment with talazoparib plus enzalutamide resulted in a 37% lower or reduced risk of imaging-based progression (blinded independent central review) or death than placebo plus enzalutamide.

Investigator-assessed rPFS was a secondary efficacy endpoint. The observed stratified hazard ratio (talazoparib+enzalutamide vs. placebo+enzalutamide) for the secondary endpoint was 0.637 (95% CI: [0.501, 0.811]; one-sided P-value: 0.0001) in favor of talazoparib+enzalutamide. Median rPFS was NE (95% CI: [30.4, NE]) months for the talazoparib+enzalutamide group vs. 30.3 months (95% CI: [24.3, NE]) months for the placebo+enzalutamide group.

OS was an alpha-protected key secondary endpoint; however, OS data were immature (31% maturity). Based on the data cutoff on 16 Aug. 2022, the number of events in the talazoparib+enzalutamide arm was 123/402 and the number of events in the placebo+enzalutamide arm was 129/402. A total of 252 deaths were observed (123 [30.6%] deaths in talazoparib+enzalutamide vs. 129 [32.0%] deaths in placebo+enzalutamide). Based on a data cutoff on 16 Aug. 2022, the median follow-up time was 28.0 months for the talazoparib+enzalutamide group and 27.1 months for the placebo+enzalutamide group. An interim analysis of OS was performed based on pre-specified O'Brien-Fleming α-spending function. The observed stratified hazard ratio (talazoparib+enzalutamide vs. placebo+enzalutamide) for OS based on 252 deaths was 0.888 (95% CI: [0.693, 1.138]; p=0.35 in favor of talazoparib+enzalutamide. Median OS was 36.4 months (95% CI: [33.5, NE/not reached]) months for the talazoparib+enzalutamide group and NE/not reached (95% CI: [33.7, NE/not reached]) months for the placebo+enzalutamide group. The interim OS results did not cross the pre-specified O'Brien-Fleming efficacy boundary.

Based on a data cutoff on 28 Mar. 2023, a total of 330 deaths were observed (156 [38.8%] deaths in talazoparib+enzalutamide versus 175 [43.25] deaths in placebo+enzalutamide). Median follow-up time was 35.8 months for the talazoparib+enzalutamide group and 34.6 months for the placebo+enzalutamide group. The observed stratified hazard ratio for OS was 0.837 (95% CI: [0.674, 1.040]; one-sided P-value: 0.00537) in favor of talazoparib+enzalutamide. Median OS was NE (95% CI: [37.3, NE] for the talazoparib+enzalutamide group and 38.2 months (95% CI: [34.1, 43.1]) for the placebo+enzalutamide group. The OS results did not cross the pre-specified O'Brien-Fleming efficacy boundary.

Objective response rate (ORR) was a secondary endpoint and it was defined as the proportion of patients with measurable soft tissue disease at baseline by BICR who had a best overall confirmed soft tissue response of CR or PR according to RECIST 1.1. The objective response rate as evaluated by BICR was 61.7% ( 74/120 events; 95% CI: [52.4, 70.4]) in the talazoparib+enzalutamide group and 43.9% ( 58/132; 95% CI: [35.3, 52.8]) in the placebo+enzalutamide group, respectively. The difference in objective response rate between the two groups was 17.7% (95% CI: [5.6, 29.9]; one-sided nominal p-value: 0.0025). Also, the CR rate was 37.5% ( 45/120) and 18.2% ( 24/132) in talazoparib+enzalutamide group and placebo+enzalutamide group, respectively. Higher rates of complete response (CR) suggested a cooperative effect of talazoparib plus enzalutamide treatment.

PSA response was also a secondary endpoint and it was defined as a decline from baseline PSA by at least 50% and confirmed by a second consecutive value at least 3 weeks later. The PSA response rate was 83.6% (95% CI: [79.6, 87.1]) in the talazoparib+enzalutamide group and 72.1% (95% CI: [67.4, 76.5]) in the placebo+enzalutamide group, respectively. The difference in PSA response was 11.5% (95% CI: [5.8,17.2]; one-sided P-value <0.0001).

Time to PSA progression was a secondary endpoint and it was the time from randomization to the first PSA progression, which was defined as a 25% increase and an absolute increase of 2 μg/L above the nadir. The observed stratified hazard ratio (talazoparib+enzalutamide vs. placebo+enzalutamide) for time to PSA progression was 0.715 (95% CI: [0.577, 0.886]; one-sided P-value: 0.0010) in favor of talazoparib+enzalutamide. Median time to PSA progression was 26.7 months (95% CI: [21.2, 30.4]) for the talazoparib+enzalutamide group and 17.5 months (95% CI: [14.1, 20.8]) for the placebo+enzalutamide group. Treatment with talazoparib plus enzalutamide prolonged time to PSA progression

Benefits of talazoparib plus enzalutamide were consistently observed across other secondary endpoints. Time to PSA progression, use of subsequent cytotoxic chemotherapy, and use of subsequent antineoplastic therapy were significantly prolonged in the talazoparib+enzalutamide arm, as shown in Table 6.

TABLE 6
Secondary Efficacy Endpoints (All-Comers Intention-to-Treat Population)

| | | | |
|---|---|---|---|
| Talazoparib + | Placebo + | | P Value |
| Enzalutamide | Enzalutamide | Hazard Ratio | (Two- |
| (N = 402) | (N = 403) | (95% CI) | Sided) |

Best overall response* - no. (%)

| | | | | |
|---|---|---|---|---|
| Complete | 45 | (37.5) | 24 | (18.2) |
| response | | | | |
| Partial response | 29 | (24.2) | 34 | (25.8) |
| Stable disease | 36 | (30.0) | 38 | (28.8) |
| Progressive | 7 | (5.8) | 30 | (22.7) |
| disease | | | | |
| Not evaluable | 3 | (2.5) | 6 | (4.5) |

| | | | |
|---|---|---|---|
| Objective response* - | 74 (61.7) | 58 (43.9) | 0.005 |
| no. (%) [95% CI] | [52.4-70.4] | [35.3-52.8] | |
| Median duration of | 20.4 | 19.8 | |
| response** - mo | [16.1-not | [12.9-not | |
| [95% CI] | reached] | reached] | |
| PSA response ≥50%*** - | 331 (83.6) | 284 (72.1) | 0.001 |
| no. (%) | [79.6-87.1] | [67.4-76.5] | |
| [95% CI] | | | |

PSA progression

| | | | | |
|---|---|---|---|---|
| Patients with | 164 | (40.8) | 177 | (43.9) |
| progression - no. | | | | |
| (%) | | | | |

| | | | | |
|---|---|---|---|---|
| Median time to | 26.7 | 17.5 | 0.72 | 0.002 |
| progression - mo | [21.2-30.4] | [14.1-20.8] | [0.58-0.89] | |
| [95% CI] | | | | |

Use of subsequent cytotoxic chemotherapy

| | | | | |
|---|---|---|---|---|
| Patients with | 83 | (20.6) | 139 | (34.5) |
| use - no. (%) | | | | |

| | | | | |
|---|---|---|---|---|
| Median time to | Not reached | Not reached | 0.49 | <0.001 |
| use - mo [95% CI] | [37.0-not | [32.3-not | [0.38-0.65] | |
| | reached] | reached] | | |

Use of subsequent antineoplastic therapy

| | | | | |
|---|---|---|---|---|
| Patients with | 114 | (28.4) | 176 | (43.7) |
| use - no. (%) | | | | |

| | | | | |
|---|---|---|---|---|
| Median time | Not reached | 28.3 | 0.54 | <0.001 |
| to use - mo | [37.0-not | [23.5-not | [0.42-0.68] | |
| | reached] | reached] | | |

Subsequent symptomatic skeletal event

| | | | | |
|---|---|---|---|---|
| Patients with | 91 | (22.6) | 93 | (23.1) |
| event - no. (%) | | | | |

| | | | | |
|---|---|---|---|---|
| Median time to | Not reached | Not reached | 0.88 | 0.41 |
| first event - mo | [Not reached] | [Not reached] | [0.66-1.18] | |

PFS2****

| | | | | |
|---|---|---|---|---|
| Patients with | 126 | (31.3) | 143 | (35.5) |
| event - no. (%) | | | | |

| | | | | |
|---|---|---|---|---|
| Median PFS2 - | 36.4 | 35.3 | 0.77 | 0.04 |
| mo | [33.5-Not | [28.6-Not | [0.61-0.98] | |
| | reached] | reached] | | |

*Only includes patients with measurable disease at baseline per blinded independent central review: talazoparib plus enzalutamide (N = 120); placebo plus enzalutamide (N = 132).
**Only includes patients with confirmed complete response or partial response: talazoparib plus enzalutamide (N = 74); placebo plus enzalutamide (N = 58).
***The number of patients with a baseline PSA value and at least one post-baseline PSA value: talazoparib plus enzalutamide (N = 396); placebo plus enzalutamide (N = 394).
****Progression-Free Survival 2 (PFS2) based on investigator assessment (time from randomization to the date of documented progression on the first subsequent antineoplastic therapy or death from any cause, whichever occurred first).

Talazoparib plus enzalutamide reduced risk of progression or death in the subgroups with deficient homologous recombination repair, non-deficient or unknown status, and non-deficient status by prospective tumor tissue testing.

In subgroup analysis by homologous recombination repair gene alteration status, the HR for ibPFS was 0.46 (95% CI, 0.30-0.70; P<0.001) in patients with a status of deficient, and 0.70 (95% CI, 0.54-0.89; P=0.004) in patients with a status of non-deficient or unknown in favor of talazoparib plus enzalutamide versus placebo plus enzalutamide. For patients with the status of deficient, median ibPFS was 27.9 months (95% CI: [16.6—NE/NR]) months for the talazoparib+enzalutamide group based on 37 events in the talazoparib+enzalutamide group of 85 patients vs. 16.4 months (95% CI: [10.9-24.6]) months for the placebo+enzalutamide group based on 49 events in the placebo+enzalutamide group of 84 patients. For patients with the status of non-deficient or unknown, median ibPFS was NR (95% CI: [27.5—NE/NR]) months for the talazoparib+enzalutamide group based on 114 events in the talazoparib+enzalutamide group of 317 patients vs. 22.5 months (95% CI: [19.1-30.5]) months for the placebo+enzalutamide group based on 142 events in the placebo+enzalutamide group of 319 patients. A clinically meaningful reduction in risk of progression or death was seen regardless of HRR status.

In exploratory subgroup analysis among patients with a HRR non-deficient status only by prospective tumor tissue testing, talazoparib plus enzalutamide resulted in a 34% lower risk of imaging-based progression or death than placebo plus enzalutamide (HR 0.66; 95% CI, 0.49-0.91; P=0.009). For patients without HRR gene alterations detected by prospective tumor tissue testing, median ibPFS by BICR was NR (95% CI: [25.8—NE/NR]) months for the talazoparib+enzalutamide group based on 70 events in the talazoparib+enzalutamide group of 198 patients vs. 22.1 months (95% CI: [16.6—NE/NR]) months for the placebo+enzalutamide group based on 96 events in the placebo+enzalutamide group of 214 patients.

In exploratory subgroup analysis, patients whose tumors had BRCA alterations had a 77% lower risk of imaging-based progression or death (HR 0.23; 95% CI, 0.10-0.53; P<0.001) and those with non-BRCA homologous recombination repair gene alterations had a 34% lower risk (HR 0.66; 95% CI. 0.39-1.12; P=0.12). Among patients without BRCA alterations or with unknown status, there was a 31% lower risk (HR 0.69; 95% CI, 0.55-0.86; P=0.001).

In addition, for DDR-deficient patients by IWRS within the all-corners cohort, the observed stratified hazard ratio (talazoparib+enzalutamide vs. placebo+enzalutamide) for BICR-assessed rPFS was 0.457 (95% CI: [0.297, 0.702]; one-sided P-value: 0.0001) in favor of talazoparib+enzalutamide. Median rPFS was 27.9 (95% CI: [16.6, NE]) months for the talazoparib+enzalutamide group vs. 16.4 months (95% CI: [10.9, 24.6]) months for the placebo+enzalutamide group. Based on a data cutoff on 28 Mar. 2023, for DDR-deficient patients by IWRS within the all-corners cohort, the observed stratified hazard ratio for OS was 0.569 (95% CI: [0.355, 0.912]; one-sided P-value: 0.0088) in favor of talazoparib+enzalutamide. Median OS was 41.9 months (95% CI: [36.4, NE]) for the talazoparib+enzalutamide group vs. 31.1 months (95% CI: [26.1, NE]) for the placebo+enzalutamide group.

For DDR-non-deficient/unknown patients by IWRS, the observed stratified hazard ratio (talazoparib+enzalutamide vs. placebo+enzalutamide) for BICR-assessed rPFS was 0.697 (95% CI: [0.544, 0.892]; P=0.004) in favor of talazoparib+enzalutamide. Median rPFS was NE (95% CI: [27.5, NE]) for the talazoparib+enzalutamide group vs. 22.5 months (95% CI: [19.1, 30.5]) months for the placebo+enzalutamide group. Based on a data cutoff on 28 Mar. 2023, for DDR-non-deficient/unknown patients per IWRS, the observed stratified hazard ratio for OS was 0.930 (95% CI: [0.729, 1.188]; one-side P-value: 0.2808) in favor of talazoparib+enzalutamide. Median OS was NE (95% CI: [37.0, NE]) for the talazoparib+enzalutamide group vs. 38.7 months (95% CI: [35.0, NE]) for the placebo+enzalutamide group.

In addition, for BRCA-mutant patients within the all-corners cohort, the observed stratified hazard ratio for OS was 0.558 (95% CI [0.263, 1.187]; one-sided P-value: 0.0622) in favor for talazoparib+enzalutamide. Median OS was 41.9 months (95% CI: [24.9, NE] for the talazoparib+enzalutamide group and 26.1 months (95% CI: [15.2, NE]) for the placebo+enzalutamide group.

For non-BRCA-mutant DDR-deficient patients within the all-corners cohort, the observed stratified hazard ratio for OS was 0.594 (95% CI: [0.322, 1.094]; one-sided P-value: 0.0454) in favor of talazoparib+enzalutamide group and 38.2 months (95% CI: [29.0, NE]) for the placebo+enzalutamide group.

For non-BRCA-mutant patients within the all-corners cohort, the observed stratified hazard ratio for OS was 0.874 (95% C: [0.696, 1.097]; one-side P-value: 0.1225) in favor of talazoparib+enzalutamide. Median OS was NE (95% C: [37.3, NE]) for the talazoparib+enzalutamide group and 38.7 months (95% CI: [35.0, 45.3]) for the placebo+enzalutamide group.

Results for secondary endpoints by deficient or non-deficient/unknown homologous recombination repair gene alteration status are in Table 7. A summary of selected subsequent systemic therapies for prostate cancer is shown in Table 8.

TABLE 7
Summary of secondary efficacy results by homologous recombination
repair status (all-comers intention-to-treat population)*

| Homologous recombination | | Homologous recombination | |
|---|---|---|---|
| repair-deficient | | repair-non-deficient/unknown | |
| Talazoparib plus | Placebo plus | Talazoparib plus | Placebo plus |
| enzalutamide | enzalutamide | enzalutamide | enzalutamide |
| (N = 85) | (N = 84) | (N = 317) | (N = 319) |

Objective response

| | | | | |
|---|---|---|---|---|
| Patients with | 33 | 26 | 87 | 106 |
| measurable | | | | |
| disease - N | | | | |

Best overall response - no. (%)

| | | | | | | | | |
|---|---|---|---|---|---|---|---|---|
| Complete | 19 | (57.6) | 5 | (19.2) | 26 | (29.9) | 19 | (17.9) |
| response | | | | | | | | |
| Partial | 7 | (21.2) | 7 | (26.9) | 22 | (25.3) | 27 | (25.5) |
| response | | | | | | | | |
| Stable | 7 | (21.2) | 8 | (30.8) | 29 | (33.3) | 30 | (28.3) |
| disease | | | | | | | | |

| | | | | | | | |
|---|---|---|---|---|---|---|---|
| Progressive | 0 | 4 | (15.4) | 7 | (8.0) | 26 | (24.5) |
| disease | | | | | | | |
| Not | 0 | 2 | (7.7) | 3 | (3.4) | 4 | (3.8) |
| evaluable | | | | | | | |

| | | | | |
|---|---|---|---|---|
| Objective | 78.8 | 46.2 | 55.2 | 43.4 |
| response - no. | [61.1-91.0] | [26.6-66.6] | [44.1-65.9] | [33.8-53.4] |
| (%) [95% CI] | | | | |

| | | |
|---|---|---|
| 2-sided P | 0.01 | 0.10 |
| value | | |

Duration of response

| | | | | |
|---|---|---|---|---|
| Patients with | 14 | 7 | 23 | 23 |
| event - N | | | | |
| Median - mo | 18.4 | 14.8 | 24.0 | 23.5 |
| [95% CI] | [10.1-not | [6.3-25.8] | [20.2-not | [15.7-not |
| | reached] | | reached] | reached] |

PSA response ≥50%

| | | | | |
|---|---|---|---|---|
| Patients | 84 | 83 | 312 | 311 |
| evaluable - N | | | | |
| Response - | 76 (90.5) | 51 (61.4) | 255 (81.7) | 233 (74.9) |
| no. (%) [95% | [82.1-95.8] | [50.1-71.9] | [77.0-85.9] | [69.7-79.6] |
| CI] | | | | |

| | | |
|---|---|---|
| 2-sided P | <0.001 | 0.04 |
| value | | |

PSA progression

| | | | | |
|---|---|---|---|---|
| Patients with | 38 | 42 | 126 | 135 |
| event - N | | | | |
| Median time to | 26.7 | 11.1 | 26.6 | 19.2 |
| progression - | [13.8-not | [9.2-19.3] | [21.2-34.1] | [15.7-24.8] |
| mo [95% CI] | reached] | | | |

| | | |
|---|---|---|
| Hazard ratio | 0.53 [0.34-0.82] | 0.78 [0.62-1.00] |
| [95% CI] | | |
| 2-sided P | 0.004 | 0.05 |
| value | | |

Use of subsequent cytotoxic chemotherapy

| | | | | |
|---|---|---|---|---|
| Patients with | 24 | 24 | 59 | 115 |
| use - N | | | | |
| Median time to | Not reached | Not reached | Not reached | Not reached |
| use - mo | [Not reached] | [28.7-not | [37.0-not | [30.4-not |
| [95% CI] | | reached] | reached] | reached] |

| | | |
|---|---|---|
| Hazard ratio | 0.71 (0.40-1.26) | 0.44 (0.32-0.61) |
| [95% CI] | | |
| 2-sided P | 0.24 | <0.001 |
| value | | |

Use of subsequent antineoplastic therapy

| | | | | |
|---|---|---|---|---|
| Patients with | 27 | 35 | 87 | 141 |
| use - N | | | | |
| Median time to | Not reached | 28.7 | Not reached | 28.3 |
| use - mo | [Not reached] | [16.7-not | [37.0-not | [23.5-not |
| [95% CI] | | reached] | reached] | reached] |

| | | |
|---|---|---|
| Hazard ratio | 0.54 (0.32-0.89) | 0.54 (0.41-0.70) |
| [95% CI] | | |
| 2-sided P | 0.01 | <0.001 |
| value | | |

Subsequent symptomatic skeletal event

| | | | | |
|---|---|---|---|---|
| Patients with | 19 | 19 | 72 | 74 |
| event - N | | | | |
| Median time to | Not reached | Not reached | Not reached | Not reached |
| first event - | [33.9-not | [Not reached] | [Not reached] | [Not reached] |
| mo [95% CI] | reached] | | | |

| | | |
|---|---|---|
| Hazard ratio | 0.72 (0.38-1.37) | 0.93 (0.67-1.29) |
| [95% CI] | | |
| 2-sided P | 0.32 | 0.67 |
| value | | |

PFS2*

| | | | | |
|---|---|---|---|---|
| Patients with | 21 | 34 | 105 | 109 |
| event - N | | | | |
| Median time to | 36.4 | 30.8 | Not reached | 35.3 |
| first event - | [36.4-not | [24.1-not | [31.6-not | [28.6-not |
| mo [95% CI] | reached] | reached] | reached] | reached] |

| | | |
|---|---|---|
| Hazard ratio | 0.47 (0.27-0.81) | 0.88 (0.67-1.14) |
| [95% CI] | | |
| 2-sided P | 0.006 | 0.33 |
| value | | |

*PFS2 based on investigator assessment (time from randomization to the date of documented progression on the first subsequent antineoplastic therapy or death from any cause, whichever occurred first).

TABLE 8
Summary of selected subsequent antineoplastic systemic therapies
for prostate cancer (all-comers safety population)

| | Talazoparib + | | Placebo + | |
|---|---|---|---|---|
| | Enzalutamide | | Enzalutamide | |
| | (N = 398) | | (N = 401) | |
| Patients taking any post-baseline | 114 | (29%) | 176 | (44%) |
| antineoplastic therapy | | | | |
| Patients taking any of the following | 102 | (26%) | 160 | (40%) |
| post-baseline antineoplastic | | | | |
| therapies with demonstrated overall | | | | |
| survival benefit | | | | |
| Cytotoxic chemotherapy | 90 | (23%) | 153 | (38%) |
| Docetaxel | 66 | (17%) | 107 | (27%) |
| Cabazitaxel | 24 | (6%) | 46 | (11%) |
| Cellular immunotherapy | 1 | (<1%) | 0 | |
| Sipuleucel-T | 1 | (<1%) | 0 | |
| Second generation androgen | 0 | | 3 | (<1%) |
| receptor inhibitors | | | | |
| Apalutamide | 0 | | 3 | (<1%) |
| Androgen biosynthesis inhibitors | 29 | (7%) | 49 | (12%) |
| Abiraterone | 29 | (7%) | 49 | (12%) |
| Single-agent PARP inhibitor | 3 | (<1%) | 11 | (3%) |
| therapies | | | | |
| Olaparib | 3 | (<1%) | 11 | (3%) |
| Radiopharmaceuticals | 13 | (3%) | 27 | (7%) |
| Lutetium* | 2 | (<1%) | 8 | (2%) |
| Radium** | 11 | (3%) | 19 | (5%) |

Data are n (%) unless otherwise noted
*Includes lutetium (177 Lu) vipivotide tetraxetan, lutetium (Lu 177), and lutetium-177.
**Includes radium, radium 223, and radium (Ra 223) dichloride.

In Table 9, a consistent treatment effect with talazoparib plus enzalutamide was seen in prespecified subgroups. The HR for all patients was based on a Cox model stratified by the randomization stratification factors. For all subgroups, the HR was based on an unstratified Cox model with treatment as the only covariate.

TABLE 9
Subgroup Analysis of ibPFS by BICR

| | | Subgroup | | | |
|---|---|---|---|---|---|
| | | Tala + | Placebo + | | 2-sided |
| | | Enza | Enza | HR (95% CI) | P value |
| Overall | | 151/402 | 191/403 | 0.63 (0.51-0.78) | <0.001 |
| Age - years | ≥70 | 93/240 | 109/240 | 0.67 (0.51-0.89) | 0.005 |
| | <70 | 58/162 | 82/163 | 0.61 (0.44-0.86) | 0.004 |
| | Asia | 37/124 | 41/117 | 0.73 (0.47-1.14) | 0.16 |
| Geographic | EU/UK | 52/150 | 73/155 | 0.59 (0.41-0.84) | 0.004 |
| region | North | 30/59 | 37/63 | 0.70 (0.43-1.13) | 0.14 |
| | America | | | | |
| | Rest of | 32/69 | 40/68 | 0.64 (0.40-1.02) | 0.06 |
| | world | | | | |
| ECOG PS | 0 | 100/259 | 130/271 | 0.67 (0.51-0.86) | 0.002 |
| | 1 | 51/143 | 61/132 | 0.62 (0.43-0.90) | 0.01 |
| Gleason Score | <8 | 34/117 | 49/113 | 0.60 (0.39-0.93) | 0.02 |
| | >8 | 115/281 | 137/283 | 0.67 (0.52-0.86) | 0.001 |
| Stage at | M0 | 64/172 | 92/185 | 0.61 (0.44-0.84) | 0.002 |
| diagnosis | M1 | 86/226 | 98/215 | 0.69 (0.51-0.92) | 0.01 |
| Progression | PSA only | 70/193 | 90/206 | 0.67 (0.49-0.92) | 0.01 |
| at entry | Imaging- | 64/150 | 69/138 | 0.67 (0.48-0.95) | 0.02 |
| | based | | | | |
| Baseline PSA | <median | 60/195 | 93/208 | 0.59 (0.42-0.81) | 0.001 |
| | ≥median | 91/206 | 97/194 | 0.67 (0.50-0.90) | 0.006 |
| Site of | Bone only | 52/169 | 63/154 | 0.59 (0.41-0.86) | 0.005 |
| metastasis | Soft tissue | 15/48 | 29/57 | 0.57 (0.30-0.107) | 0.07 |
| | only | | | | |
| | Bone and | 82/180 | 98/188 | 0.71 (0.53-0.95) | 0.02 |
| | soft tissue | | | | |
| HRR status per | Deficient | 37/85 | 49/84 | 0.48 (0.31-0.74) | 0.001 |
| randonmization | Non- | 114/317 | 142/319 | 0.69 (0.54-0.89) | 0.004 |
| stratification | deficient/ | | | | |
| | unknown | | | | |
| Prior | Yes | 42/109 | 58/110 | 0.56 (0.38-0.83) | 0.004 |
| abiraterone or | No | 109/293 | 133/293 | 0.68 (0.53-0.88) | 0.003 |
| docetaxel* | | | | | |
| BRCA1/2 | BRCA1/2- | 8/27 | 22/32 | 0.23 (0.10-0.53) | 0.0002 |
| status and | altered, | | | | |
| HRR gene | HRR | | | | |
| alteration | deficient | | | | |
| status | Non- | 29/58 | 27/52 | 0.66 (0.39-1.12) | 0.12 |
| | BRCA1/2- | | | | |
| | latered, | | | | |
| | HRR | | | | |
| | deficient | | | | |
| | Non- | 143/375 | 169/371 | 0.69 (0.55-0.86) | 0.0011 |
| | BRCA1/2- | | | | |
| | altered or | | | | |
| | unknown, | | | | |
| | intention- | | | | |
| | to-treat | | | | |
| Prospective | HRR- | 36/83 | 47/80 | 0.45 (0.29-0.69) | 0.0002 |
| tumour testing | deficient | | | | |
| | HRR-non- | 70/198 | 96/214 | 0.66 (0.49-0.91) | 0.0092 |
| | deficient | | | | |
| | HRR- | 45/121 | 48/109 | 0.79 (0.52-1.18) | 0.25 |
| | unknown | | | | |

*^{a}Includes two patients in each treatment arm who received prior orteronel.

As shown in Table 9, all enrolled patients (N=805) had prospective tumor tissue HRR test results. Overall, baseline characteristics were relatively well-balanced between treatment groups and by HRR status; however, in the younger group (age <65 y), there were more HRR+ (29.6%) than HRR−/unknown (19.3%) pts, and HRR+pateints had evidence of more aggressive disease. Treatment with talazoparib plus enzalutamide improved ORR, and prolonged time to PSA progression; time to initiation of cytotoxic chemotherapy; and PFS2 vs placebo plus enzalutamide, with benefit seen in both HRR+ and HRR−/unknown subgroups.

Tables 10 and 11 below show forest plots data of OS by baseline characteristics and by DDR subgroups based on the following definition: prospective tumor tissue and prospective ctDNA samples; prospective samples plus retrospective ctDNA samples; tumor tissue samples only; and retrospective ctDNA samples only.

TABLE 10
OS Analysis by Baseline Subgroups

| | | Subgroup | | | | | | |
|---|---|---|---|---|---|---|---|---|
| | | Tala + | | Placebo + | | | HR (95% | 1-sided |
| | | Enza | | Enza | | | CI) | P value |
| | | Sensitivity Analysis | | | | | | |
| | | N (E) | | | | Median (mo) | | |
| Overall | | 402 | (156) | 403 | (174) | NE/38.2 | 0.837 | 0.0538 |
| | | | | | | | (0.674, | |
| | | | | | | | 1.040) | |
| Age - | ≥70 | 240 | (104 | 240 | (190) | 37.6/33.9 | 0.779 | 0.0320 |
| years | | | | | | | (0.599, | |
| | | | | | | | 1.1015) | |
| | <70 | 162 | (52) | 163 | (55) | NE/45.3 | 0.955 | 0.4054 |
| | | | | | | | (0.652, | |
| | | | | | | | 1.397) | |
| Geographic | Asia | 124 | (39) | 117 | (38) | NE/43.1 | 0.884 | 0.2957 |
| region | | | | | | | (0.566, | |
| | | | | | | | 1.383) | |
| | EU/GBR | 150 | (57) | 155 | (69) | 41.9/38.2 | 0.799 | 0.1043 |
| | | | | | | | (0.562, | |
| | | | | | | | 1.135) | |
| | North | 59 | (32) | 63 | (37) | 31.4/27.4 | 0.837 | 0.2304 |
| | America | | | | | | (0.521, | |
| | | | | | | | 1.345) | |
| | Rest of | 69 | (28) | 68 | (30) | NE/38.0 | 0.920 | 0.3757 |
| | world | | | | | | (0.549, | |
| | | | | | | | 1.541) | |
| ECOG | 0 | 259 | (98) | 271 | (106) | NE/39.8 | 0.915 | 0.2627 |
| PS | | | | | | | (0.695, | |
| | | | | | | | 1.204) | |
| | 1 | 143 | (58) | 132 | (68) | NE/31.1 | 0.719 | 0.0318 |
| | | | | | | | (0.506, | |
| | | | | | | | 1.021) | |
| Gleason | <8 | 117 | (43) | 113 | (46) | NE/38.4 | 0.907 | 0.3233 |
| Score | | | | | | | (0.598, | |
| | | | | | | | 1.375) | |
| | >8 | 281 | (111) | 283 | (125) | 41.9/38.0 | 0.827 | 0.0724 |
| | | | | | | | (0.640, | |
| | | | | | | | 1.068) | |
| Stage at | M0 | 172 | (64) | 185 | (78) | 41.9/38.2 | 0.849 | 0.1652 |
| diagnosis | | | | | | | (0.610, | |
| | | | | | | | 1.068) | |
| | M1 | 226 | (92) | 215 | (94) | NE/38.7 | 0.858 | 0.1488 |
| | | | | | | | (0.643, | |
| | | | | | | | 1.144) | |
| Progression | PSA only | 193 | (66) | 206 | (83) | NE/39.4 | 0.783 | 0.0694 |
| at entry | | | | | | | (0.566, | |
| | | | | | | | 1.083) | |
| | RP with or | 150 | (71) | 138 | (73) | 37.2/32.2 | 0.814 | 0.1087 |
| | w/o PSA | | | | | | (0.587, | |
| | prog. | | | | | | 1.129) | |
| Baseline | <median | 194 | (58) | 207 | (65) | NE/43.1 | 0.930 | 0.3434 |
| PSA | | | | | | | (0.652, | |
| | | | | | | | 1.325) | |
| | ≥median | 207 | (98) | 195 | (108) | 33.0/27.9 | 0.750 | 0.0194 |
| | | | | | | | (0.570, | |
| | | | | | | | 0.986) | |
| Site of | Bone only | 168 | (51) | 154 | (72) | NE/37.5 | 0.583 | 0.0014 |
| metastasis | | | | | | | (0.407, | |
| | | | | | | | 0.835) | |
| | Soft tissue | 48 | (12) | 57 | (14) | NE/NE | 1.068 | 0.5656 |
| | only | | | | | | (0.492, | |
| | | | | | | | 2.318) | |
| | Bone and | 181 | (91) | 188 | (88) | 33.0/33.9 | 1.012 | 0.5310 |
| | soft tissue | | | | | | (0.754, | |
| | | | | | | | 1.357) | |
| DDR | Deficient | 85 | (31) | 54 | (41) | 41.9/31.1 | 0.585 | 0.0119 |
| status by | | | | | | | (0.365, | |
| IWRS | | | | | | | 0.936) | |
| | Non- | 317 | (125) | 319 | (133) | NE/38.7 | 0.926 | 0.2691 |
| | deficient/ | | | | | | (0.725, | |
| | unknown | | | | | | 1.182) | |
| Prio | Yes | 109 | (46) | 110 | (50) | 37.2/34.4 | 0.850 | 0.2125 |
| Taxane | | | | | | | (0.569, | |
| or NHT | | | | | | | 1.268) | |
| by IWRS | No | 293 | (110) | 293 | (124) | NE/38.7 | 0.843 | 0.0956 |
| | | | | | | | (0.652, | |
| | | | | | | | 1.090) | |

TABLE 11
OS Analysis by DDR Subgroups

| | Subgroup | | | | | | |
|---|---|---|---|---|---|---|---|
| | Tala + | | Placebo + | | | HR (95% | 1-sided |
| | Enza | | Enza | | | CI) | P value |
| | Subgroup | | | | | | |
| | N (E) | | | | Median (mo) | | |
| All Patiens | 402 | (156) | 403 | (174) | NE/38.2 | 0.837 | 0.0538 |
| | | | | | | (0.674, | |
| | | | | | | 1.040) | |
| DDR-decifient by | 85 | (30) | 82 | (41) | 41.9/30.8 | 0.530 | 0.0040 |
| prospective tumor | | | | | | (0.330, | |
| tissue and blood | | | | | | 0.854) | |
| Non-DDR-deficient by | 207 | (82) | 219 | (96) | NE/38.0 | 0.883 | 0.2028 |
| prospective | | | | | | (0.657, | |
| | | | | | | 1.185) | |
| DDR-unknown by | 110 | (44) | 102 | (52) | NE/45.3 | 1.148 | 0.7310 |
| prospective tumor | | | | | | (0.739, | |
| tissue and blood | | | | | | 1.784) | |
| DDR-deficient by | 107 | (42) | 107 | (52) | 41.9/34.4 | 0.678 | 0.0305 |
| prospective plus | | | | | | (0.450, | |
| retrospective plasma | | | | | | 1.021) | |
| Non-DDR-deficient by | 270 | (108) | 277 | (114) | NE/38.7 | 0.980 | 0.4413 |
| prospective plus | | | | | | (0.753, | |
| retrospective plasma | | | | | | 1.276 | |
| DDR-unknown by | 25 | (6) | 19 | (8) | NE/30.5 | 0.471 | 0.0776 |
| prospective plus | | | | | | (0.163, | |
| retrospective plasma | | | | | | 1.363) | |
| DDR-deficiencies | 107 | (42) | 107 | (52) | 41.9/34.4 | 0.678 | 0.0305 |
| prospective plus | | | | | | (0.450, | |
| retrospective plasma | | | | | | 1.021) | |
| plus saliva | | | | | | | |
| Non-DDR- | 270 | (108) | 277 | (114) | NE/38.7 | 0.980 | 0.4413 |
| deficiencies by | | | | | | (0.753, | |
| prospective plus | | | | | | 1.276) | |
| retrospective plasma | | | | | | | |
| plus saliva | | | | | | | |
| DDR-unknown by | 25 | (6) | 19 | (8) | NE/30.5 | 0.471 | 0.0776 |
| prosepective plus | | | | | | (0.163, | |
| retrospective plasma | | | | | | 1.363) | |
| plus saliva | | | | | | | |
| DDR-deficient by | 83 | (29) | 80 | (40) | 41.9/30.8 | 0.521 | 0.0035 |
| tumor tissue | | | | | | (0.321, | |
| | | | | | | 0.844) | |
| Non-DDR-decifient by | 198 | (78) | 314 | (96) | NE/37.5 | 0.847 | 0.1375 |
| tumor tissue | | | | | | (0.628, | |
| | | | | | | 1.142) | |
| DDR-unknown by | 121 | (49) | 109 | (38) | NE/45.3 | 1.240 | 0.8383 |
| tumor tissue | | | | | | (0.809, | |
| | | | | | | 1.900) | |
| DDR-deficient by | 100 | (39) | 108 | (57) | NE/31.1 | 0.665 | 0.0245 |
| ctDNA | | | | | | (0.441, | |
| | | | | | | 1.001) | |
| Non-DDR-deficient by | 241 | (98) | 239 | (93) | 41.9/43.1 | 1.039 | 0.6051 |
| ctDNA | | | | | | (0.782, | |
| | | | | | | 1.381) | |
| DDR-unknown by | 61 | (19) | 56 | (24) | NE/38.0 | 0.601 | 0.0504 |
| ctDNA | | | | | | (0.332, | |
| | | | | | | 1.109) | |

### Safety

A comprehensive safety analysis is ongoing. To date the safety profile of talazoparib in combination with enzalutamide for mRPC is generally consistent with the known safety profiles of each medicine.

The safety population consisted of 799 patients who were treated with at least one dose of study treatment; 398 patients were treated with talazoparib+enzalutamide and 401 patients were treated with placebo+enzalutamide.

Median duration of treatment was 19.8 months for talazoparib and 22.2 months for enzalutamide in the talazoparib+enzalutamide group, and 16.1 months for placebo and 16.6 months for enzalutamide in the placebo+enzalutamide. Median relative dose intensities in the talazoparib+enzalutamide group were 83.5% for talazoparib and 100% for enzalutamide.

The median duration of talazoparib treatment was 86 weeks and the median duration of placebo was 70 weeks. The median duration of enzalutamide treatment was 97 weeks for the talazoparib+enzalutamide group and 72 weeks for the placebo+enzalutamide group. See Table 12 below.

TABLE 12
Dosing Exposure (Safety Population)

| | Talazoparib + | Placebo + |
|---|---|---|
| | Enzalutamide | Enzalutamide |
| | (N = 398) | (N = 401) |
| Talazoparib/placebo | 397 | 400 |
| Median duration of treatment | 86.00 | 69.86 |
| (weeks) | | |
| Median average daily dose | 0.38 | 0.50 |
| administered (mg/day) | | |
| Median relative dose intensity (%) | 83.54 | 100 |
| Enzalutamide | 398 | 401 |
| Median duration of treatment | 96.57 | 72 |
| (weeks) | | |
| Median average daily dose | 159.79 | 160 |
| administered (mg/day) | | |
| Median relative dose intensity (%) | 99.87 | 100 |

The numbers of patients with treatment-emergent AEs (TEAEs) and serious adverse events (SAEs) were summarized in Table 13. Treatment-emergent is defined as the time between the first dose of study treatment to 28 days after the last dose of the last study treatment, or before any new antineoplastic therapy, whichever occurs first.

TABLE 13
Summary of Adverse Events (All-Comers Safety Population)

| | Talazoparib + | | | | Placebo + | | | |
|---|---|---|---|---|---|---|---|---|
| | Enzalutamide | | | | Enzalutamide | | | |
| | (N = 398) | | | | (N = 401) | | | |
| | All Grades | | Grade ≥3 | | All Grades | | Grade ≥3 | |
| Any adverse event | 392 | (98.5%) | 299 | (75.1) | 379 | (94.5%) | 181 | (45.1) |
| Treatment-related | 357 | (89.7) | 234 | (58.8) | 279 | (69.6) | 71 | (17.7) |
| adverse event | | | | | | | | |
| maximum Grade 3 or 4 | 286 | (71.9%) | — | | 163 | (40.6%) | — | |
| AEs | | | | | | | | |
| maximum Grade 5 AEs | 13 | (3.3%)* | — | | 18 | (4.5%)** | — | |
| Treatment-related Grade | 0 | | — | | 2 | (0.5) | — | |
| 5 AEs | | | | | | | | |
| any serious adverse | 157 | (39.4%) | 145 | (36.4) | 107 | (26.7%) | 94 | (23.4) |
| event (SAE) | | | | | | | | |
| Serious and treatment- | 78 | (19.6) | 68 | (17.1) | 12 | (3.0) | 11 | (2.7) |
| related adverse event | | | | | | | | |

Adverse event resulting in permanent drug discontinuation from:

| | | | | | |
|---|---|---|---|---|---|
| ONLY | 39 | (9.8%) | — | 8 | (2.0%) |
| talazoparib/placebo | | | | | |
| ONLY enzalutamide | 6 | (1.5%) | — | 3 | (0.7%) |
| BOTH | 37 | (9.3%) | — | 41 | (10.2%) |
| talazoparib/placebo and | | | | | |
| enzalutamide | | | | | |

TEAE resulting in dose reduction on:

| | | | | |
|---|---|---|---|---|
| ONLY | 201 | (50.5%) | 21 | (5.2%) |
| talazoparib/placebo | | | | |
| ONLY enzalutamide | 44 | (11.1%) | 24 | (6.0%) |
| dose reduction on BOTH | 22 | (5.5%) | 8 | (2.0%) |
| talazoparib/placebo and | | | | |
| enzalutamide | | | | |

*None were considered treatment-related.
**Two were considered treatment-related.

The most frequent treatment-emergent AEs of any Grade (G) and causality experienced in ≥10% patients in either arm were summarized in Table 14 below and arranged in a descending order based on the frequency of events in the talazoparib+enzalutamide group.

TABLE 14
TEAEs in ≥10% Patients - All Causalities (Safety Population)

| | Talazoparib + | | | | Placebo + | | | |
|---|---|---|---|---|---|---|---|---|
| | Enzalutamide | | | | Enzalutamide | | | |
| | (N = 398) | | | | (N = 401) | | | |
| Preferred Term | All Grade | | Grade ≥3 | | All Grade | | Grade ≥3 | |
| Anemia | 262 | (65.8%) | 185 | (46.5%) | 70 | (17.5%) | 17 | (4.2%) |
| Neutrophil count | 142 | (35.7%) | 73 | (18.3%) | 28 | (7.0%) | 6 | (1.5%) |
| decreased | | | | | | | | |
| Fatigue | 134 | (33.7%) | 16 | (4.0%) | 118 | (29.4%) | 8 | (2.0%) |
| Platelet count decreased | 98 | (24.6%) | 29 | (7.3%) | 14 | (3.5%) | 4 | (1.0%) |
| (thrombocytopenia) | | | | | | | | |
| Back pain | 88 | (22.1%) | 10 | (2.5%) | 72 | (18.0%) | 4 | (1.0%) |
| White blood cell count | 88 | (22.1%) | 25 | (6.3%) | 18 | (4.5%) | 0 | |
| decreased (leukopenia) | | | | | | | | |
| Decreased appetite | 86 | (21.6%) | 5 | (1.3%) | 63 | (15.7%) | 4 | (1.0%) |
| Nausea | 82 | (20.6%) | 2 | (0.5%) | 50 | (12.5%) | 3 | (0.7%) |
| Constipation | 72 | (18.1%) | 1 | (0.3%) | 68 | (17.0%) | 2 | (0.5%) |
| Fall | 71 | (17.8%) | 9 | (2.3%) | 59 | (14.7%) | 8 | (2.0%) |
| Arthralgia | 58 | (14.6%) | 2 | (0.5%) | 79 | (19.7%) | 2 | (0.5%) |
| Asthenia | 57 | (14.3%) | 11 | (2.8%) | 38 | (9.5%) | 3 | (0.7%) |
| Diarrhoea | 57 | (14.3%) | 1 | (0.3%) | 55 | (13.7%) | 0 | |
| Hypertension | 55 | (13.8%) | 21 | (5.3%) | 62 | (15.5%) | 30 | (7.5%) |
| Dizziness | 48 | (12.1%) | 4 | (1.0%) | 23 | (5.7%) | 2 | (0.5%) |
| Hot flush | 47 | (11.8%) | 0 | | 53 | (13.2%) | 0 | |
| Lymphocyte count | 45 | (11.3%) | 20 | (5.0%) | 20 | (5.0%) | 4 | (1.0%) |
| decreased (lymphopenia) | | | | | | | | |
| Oedema peripheral | 42 | (10.6%) | 0 | | 23 | (5.7%) | 0 | |
| Dyspnoea | 41 | (10.3%) | 2 | (0.5%) | 25 | (6.2%) | 1 | (0.2%) |
| Weight decreased | 40 | (10.1%) | 2 | (0.5%) | 33 | (8.2%) | 3 | (0.7%) |

The most frequent SAEs observed in the study were anemia, which occurred in 55 (13.8%) patients in the talazoparib+enzalutamide group and 1 (0.2%) patient in the placebo+enzalutamide group; haematuria, which occurred in 10 (2.5%) patients in the the talazoparib+enzalutamide group and 4 (1.0%) patients in the placebo+enzalutamide group; and urinary tract infection, which occurred in 9 (2.3%) patients in the talazoparib+enzalutamide group and 3 (0.7%) patients in the placebo+enzalutamide group. See Table 15 below.

TABLE 15
SAEs in ≥2% Patients - All Causalities (Safety Population)

| | Talazoparib + | | Placebo + |
|---|---|---|---|
| | Enzalutamide | | Enzalutamide |
| Preferred Term | (N = 398) | | (N = 401) |
| Anemia | 55 | (13.8%) | 1 (0.2%) |
| Haematuria | 10 | (2.5%) | 4 (1.0%) |
| Urinary tract infection | 9 | (2.3%) | 3 (0.7%) |

There were 14 (3.5%) deaths within 28 days after last dose of study treatment in the talazoparib+enzalutamide group and 20 (5.0%) deaths within 28 days after last dose in the placebo+enzalutamide group, respectively.

The most common all-cause treatment-emergent adverse events in the are shown in Table 16.

TABLE 16
Most Common All-Cause Treatment-Emergent Adverse Events

| | Talazoparib + | | | Placebo + | | |
|---|---|---|---|---|---|---|
| | Enzalutamide | | | Enzalutamide | | |
| | (N = 398) | | | (N = 401) | | |
| | G1-2 | G3 | G4 | G1-2 | G3 | G4 |
| Preferred Term | (%) | (%) | (%) | (%) | (%) | (%) |
| Anemia | 19.3 | 43.2 | 3.3 | 13.2 | 4.2 | = |
| Neutropenia | 17.3 | 17.1 | 1.3 | 5.5 | 1.0 | 0.5 |
| Fatigue | 29.6 | 4.0 | — | 27.4 | 2.0 | — |
| Thrombocytopenia | 17.3 | 5.0 | 2.3 | 2.5 | 0.7 | 0.2 |
| Leukopenia | 15.8 | 6.3 | — | 4.5 | — | — |
| Back pain | 19.6 | 2.5 | — | 17.0 | 1.0 | — |
| Decreased | 20.4 | 1.3 | — | 14.7 | 1.0 | — |
| appetite | | | | | | |
| Nausea | 20.1 | 0.5 | | 11.7 | 0.7 | |

The most common all-cause adverse events in the talazoparib+enzalutamide group (30% of patients) were anemia, neutropenia, and fatigue.

The three most common nonhematologic TEAEs were fatigue (33.7%; 4.0% G3), back pain (22.1%; 2.5% G3), and decreased appetite (21.6%; 1.3% G3).

The three most common hematologic all-cause TEAEs and associated dose modifications were anemia, neutropenia, and thrombocytopenia. Details provided below in Table 17.

TABLE 17
Most Common Hematologic All-Cause Adverse Events
in the Talazoparib + Enzalutamide Group

| | | | | | | G ≥ 3 median | Led to Tala dose |
|---|---|---|---|---|---|---|---|
| | | | | | | time to | interruption/ |
| | | | | | Any | onset/ | reduction/ |
| Tala + Enza | G1 | G2 | G3 | G4 | G | duration | discontinuation |
| (N = 398) | (%) | (%) | (%) | (%) | (%) | (mo, d)^{a} | (%)^{a} |
| Anemia | 6.0 | 13.3 | 43.2 | 3.3 | 65.8 | 3.3/16 | 44.2/43.2/8.3 |
| Neutropenia | 3.0 | 14.3 | 17.1 | 1.3 | 35.7 | 2.3/12 | 13.6/15.1/3.3 |
| Thrombocytopenia | 10.3 | 7.0 | 5.0 | 2.3 | 24.6 | 2.3/19 | 7.8/5.5/0.5 |

^{a}Cluster terms used for anemia, neutropenia, and thrombocytopenia.

The most common Grade 3-4 adverse events (10% of patients) were anemia (46.5%) and neutropenia (18.3%) in the talazoparib+enzalutamide group.

To ensure optimal dosing of talazoparib at the individual patient level, the protocol did not require dose modification of talazoparib until anemia was Grade (G) ≥3. The median time to onset of first G≥3 anemia was 3.3 months. Following Grade 3-4 anemia, the protocol required dose hold, and then dose reduction of talazoparib. At baseline, 49.0% had G1-2 anemia. Per protocol, 43.2% had anemia leading to dose reduction (with or without transfusion); 20.6% had recurrent G3-4 anemia and 8.3% discontinued talazoparib due to anemia.

There were more dose interruptions and reductions due to adverse events in the talazoparib+enzalutamide group than in the placebo+enzalutamide group (Table 13). The dose of talazoparib was interrupted due to adverse events in 300 (75.4%) patients compared with 94 (23.4%) patients who had a dose interruption of placebo. The talazoparib dose was reduced due to adverse events in 223 (56.0%) patients and the placebo dose was reduced due to adverse events in 29 (7.2%) patients. The most common adverse events leading to a dose reduction of talazoparib were anemia (179 [45.0%] patients), neutropenia (62 [15.6%]), thromboctyopenia (23 [5.8%]), and leukopenia (9 [2.3%]). The three most common adverse events leading to a dose reduction of talazoparib were anemia (43.2%), neutropenia ([5.1%]), and thromboctyopenia (5.5%). Discontinuation of talazoparib occurred in 76 (19.1%) patients and 49 (12.2%) patients discontinued placebo due to adverse events. The most common adverse events leading to discontinuation of talazoparib were anemia (33 [8.3%]) and neutropenia (13 [3.3%]). Discontinuation rates of enzalutamide due to adverse events were 10.8% (talazoparib+enzalutamide group) versus 11.0% (placebo+enzalutamide arm).

TABLE 18
Summary of Dose Modifications due to Adverse
Events (All-Comers Safety Population)

| | |
|---|---|
| Talazoparib plus | Placebo plus |
| enzalutamide | enzalutamide |
| (N = 398) | (N = 401) |
| no. (%) | no. (%) |

Dose interruptions

| | | | | |
|---|---|---|---|---|
| Talazoparib/placebo¹ | 300 | (75.4) | 94 | (23.4) |
| Enzalutamide² | 196 | (49.2) | 85 | (21.2) |
| Dose reductions | | | | |
| Talazoparib/placebo³ | 223 | (56.0) | 29 | (7.2) |
| Enzalutamide⁴ | 66 | (16.6) | 32 | (8.0) |
| Permanent discontinuations | | | | |
| Talazoparib/placebo⁵ | 76 | (19.1) | 49 | (12.2) |
| Enzalutamide⁶ | 43 | (10.8) | 44 | (11.0) |

¹Includes dose interruptions of talazoparib/placebo only plus interruptions of both talazoparib/placebo and enzalutamide
²Includes dose interruptions of enzalutamide only plus interruptions of both talazoparib/placebo and enzalutamide
³Includes dose reductions of talazoparib/placebo only plus reductions of both talazoparib/placebo and enzalutamide
⁴Includes dose reductions of enzalutamide only plus dose reductions of both talazoparib/placebo and enzalutamide
⁵Includes permanent discontinuations of talazoparib/placebo only plus discontinuations of both talazoparib/placebo and enzalutamide
⁶Includes permanent discontinuations of enzalutamide only plus discontinuations of both talazoparib/placebo and enzalutamide

TABLE 19
Summary of Dose Modifications and Discontinuations
of Talazoparib due to Anemia, Neutopenia,
and Thrombocytopenia (Safety Population)

| | Talazoparib + Enzalutamide (N = 398) | | | | | |
|---|---|---|---|---|---|---|
| | Interruption | | Reduction | | Discontinuation | |
| | n (%) | | n (%) | | n (%) | |
| Anemia | 176 | (44.2) | 172 | (43.2) | 33 | (8.3) |
| Neutropenia | 196 | (49.2) | 85 | (21.2) | 13 | (3.3) |
| Thrombocytopenia | 31 | (7.8) | 22 | (5.5) | 2 | (0.5) |

In the talazoparib+enzalutamide group, there was one instance of myelodysplastic syndrome during the safety reporting period and one instance of acute myeloid leukemia during the follow-up period (none in the placebo+enzalutamide arm). Venous embolic and thrombotic events were reported in 16 (4.0%) patients in the talazoparib+enzalutamide group, including 10 (2.5%) patients with pulmonary embolism (Grade ≥3 in 9 patients), and in 3 (0.7%) patients in the placebo+enzalutamide arm, all of which were Grade ≥3 pulmonary embolisms.

The safety profile of talazoparib plus enzalutamide was consistent with individual profiles, and TEAEs were generally managed through dose modifications and supportive measures. Talazoparib 0.5 mg QD plus enzalutamide 160 mg QD was generally manageable, with dose modifications of talazoparib and/or standard supportive care. Anemia was the most common TEAE and led to discontinuation of talazoparib in 8.3% of patients. Table 20 shows the summary of supportive care measures for the treatment of anemia. G≥3 hematologic AEs typically occurred within 6 months of starting therapy and were short-lived, and reduction in hemoglobin levels was most pronounced early in treatment, followed by rebounding of hemoglobin levels.

TABLE 20
Summary of supportive care measures for the
treatment of anemia (safety population)

| | Talazoparib + | |
|---|---|---|
| | Enzalutamide | |
| Supportive Care | (N = 398) | |
| Patients receiving at least 1 | 53 | (13.1) |
| hematologic supportive treatment | | |
| Erythropoietin-stimulating agents* | 33 | (8.3) |
| Granulocye-stimulating factors ** | 30 | (7.5) |
| Platelet-stimulating factors* | 6 | (1.5) |

All values are shown as n (%). The following medications are considered as hematologic supportive treatments and included in the table:
*Epoetin alpha, darbepoetin alpha, epoetin theta, erythropoietin, erythropoietin human, epoetin zeta.
** Filgrastim, lenograstim, lipegfilgrastim, granulocyte colony-stimulating factor, pegylated granulocyte colony-stimulating factor, granulocyte-macrophage colony-stimulating factor, filgrastim-sndz
***Oprelvekin.

### Patient-Reported Outcomes

Median time to definitive clinically meaningful deterioration in global health status/quality of life (GHS/QoL) was significantly longer in the talazoparib+enzalutamide group (30.8 months [95% CI, 27-39.6]) based on 138 events than the placebo+enzalutamide arm (25 months [95% CI, 22.9-30.4] based on 146 events; HR 0.78 [95% CI, 0.62-0.99]; P=0.04). Definitive clinically meaningful deterioration was defined as a ≥10-point decrease from baseline and no subsequent observations with <10-point decrease from baseline assessed by the European Organisation for Research and Treatment of Cancer cancer-specific global health questionnaire (EORTC QLQ-C30). Talazoparib plus enzalutamide significantly prolonged time to definitive clinically meaningful deterioration in GHS/QoL.

Overall estimated mean change in global health status/quality of life significantly favored placebo plus enzalutamide (−2.5; 95% CI, −4.6--0.5; P=0.014).

### CONCLUSION

The all-corners cohort met its primary endpoint. Talazoparib demonstrated clinical benefit in combination with enzalutamide in metastatic mCRPC. Talazoparib in combination with enzalutamide statistically significantly prolonged rPFS when compared with placebo in combination with enzalutamide in mCRPC patients unselected for DDR status. The robust, highly consistent efficacy was demonstrated in men both with and without homologous recombination repair gene mutations. Across the all-corners population and pre-specified subgroups, talazoparib plus enzalutamide resulted in clinically meaningful and statistically significant benefits over enzalutamide plus placebo. Benefits were consistently observed across the secondary endpoints. In patients with measurable disease who received talazoparib plus enzalutamide, the complete response rate was double that with placebo plus enzalutamide. Results from the primary analysis of the all-corners population of the TALAPRO-2 trial support the use of talazoparib plus enzalutamide as a first-line treatment in patients with metastatic castration-resistant prostate cancer unselected for homologous recombination repair gene alterations.

First-line talazoparib plus enzalutamide significantly reduced the risk of disease progression or death by 37% versus placebo plus enzalutamide for molecularly unselected patients with metastatic castration-resistant prostate cancer.

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

1. A method of treating metastatic castration-resistant prostate cancer in a subject in need thereof, comprising: 1) orally administering to the subject talazoparib, or a pharmaceutically acceptable salt thereof, once daily; and 2) orally administering to the subject enzalutamide, or a pharmaceutically acceptable salt thereof, once daily, wherein the administration of the talazoparib, or a pharmaceutically acceptable salt thereof, and the administration of the enzalutamide, or a pharmaceutically acceptable salt thereof, increases survival in the subject.

2. The method of claim 1, wherein the metastatic castration-resistant prostate cancer is metastatic castration-resistant prostate cancer with or without homologous recombination repair (HRR) gene mutations.

3. The method of claim 1, wherein the subject has not received: 1) systemic cancer treatment for non-metastatic castration-resistant prostate cancer or metastatic castration-resistant prostate cancer; 2) treatment with an androgen receptor signaling inhibitor, a PARP inhibitor, cyclophosphamide, or mitoxantrone for prostate cancer; or 3) treatment with platinum-based chemotherapy within 6 months or any history of disease progression on platinum-based therapy within 6 months.

4. The method of claim 1, wherein the subject is additionally receiving a gonadotropin releasing hormone analog or has had a bilateral orchiectomy.

5. The method of claim 4, wherein the gonadotropin releasing hormone analog is a gonadotropin releasing hormone agonist or a gonadotropin releasing hormone antagonist.

6. The method of claim 1, wherein the subject is unselected for DNA damage response (DDR) mutation status.

7. The method of claim 1, wherein survival is radiographic progression free survival.

8. The method of claim 7, wherein radiographic progression free survival is increased as compared to a subject who has received enzalutamide, or a pharmaceutically acceptable salt thereof, and placebo.

9. The method of claim 1, wherein survival is overall survival.

10. The method of claim 9, wherein overall survival is increased as compared to a subject who has received enzalutamide, or a pharmaceutically acceptable salt thereof, and placebo.

11. The method of claim 1, wherein survival is a significantly reduced risk of disease progression or death, as compared to a subject who has received enzalutamide, or a pharmaceutically acceptable salt thereof, and placebo.

12. The method of claim 11, wherein the significantly reduced risk of disease progression or death is at least a 37% reduction, as compared to a subject who has received enzalutamide, or a pharmaceutically acceptable salt thereof, and placebo.

13. The method of claim 1, wherein the talazoparib, or pharmaceutically acceptable salt thereof, is administered at a dosage equivalent to about 0.1 mg, about 0.25 mg, about 0.35 mg or about 0.5 mg once daily of talazoparib free base.

14. The method of claim 13, wherein the talazoparib, or pharmaceutically acceptable salt thereof, is administered at a dosage equivalent to about 0.35 mg once daily of talazoparib free base and further wherein the subject has moderate renal impairment.

15. The method of claim 1, wherein the talazoparib, or pharmaceutically acceptable salt thereof, is talazoparib tosylate.

16. The method of claim 1, wherein the enzalutamide, or pharmaceutically acceptable salt thereof, is administered at a dosage equivalent to about 160 mg once daily of enzalutamide free base.

17. The method of claim 16, wherein the dosage of enzalutamide, or a pharmaceutically acceptable salt thereof, is reduced, if enzalutamide, or a pharmaceutically acceptable salt thereof, is dosed concomitantly with a strong CYP2C8 inhibitor.

18. The method of claim 17, wherein the dose of enzalutamide, or a pharmaceutically acceptable salt thereof, is reduced to 80 mg once daily.

19. The method of claim 16, wherein the dose of enzalutamide, or a pharmaceutically acceptable salt thereof, is increased if the enzalutamide is dosed concomitantly with a CYP3A4 inducer.

20. The method of claim 19, wherein the dose of enzalutamide, or a pharmaceutically acceptable salt thereof, is increased to 240 mg daily.

21. The method of claim 1, wherein the enzalutamide, or a pharmaceutically acceptable salt thereof, is a free base.

22. The method of claim 1, wherein the subject is a human.

23. The method of claim 22, wherein the human is an adult.
